(19) United States
(12) Patent Application Publication
(10) Pub. No.: US 2004/0029891 A1
(43) Pub. Date: Feb. 12, 2004

(54) Use of PDE5 inhibitors in the treatment of polycystic ovary syndrome

(75) Inventors: Mahmoud Nizar Ghazzi, Ann Arbor, MI (US); Nandan Parmanand Koppiker, Sandwich (GB); Simon Lempriere Westbrook, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/355,418

(22) Filed: Sep. 2, 2003

Related U.S. Application Data
(60) Provisional application No. 60/355,038, filed on Feb. 7, 2002.

(51) Int. Cl. A61K 31/519

(52) U.S. Cl. 514/252.16; 514/262.1

(57) ABSTRACT

The present invention relates to the use of a pyrazolopyrimidinone PDE5 inhibitor such as sildenafil for the treatment of polycystic ovary syndrome.

### FIELD OF THE INVENTION

The present invention relates to the treatment of polycystic ovary syndrome (sometimes referred to as PCOS) and to compounds and compositions for such treatment, as well as the uses thereof of said compounds and compositions.

In particular the present invention relates to the use of pyrazolopyrimidinone inhibitors of cyclic guanosine 3′,5′-monophosphate phosphodiesterase type five (PDE5 or PDE V) for treatment of PCOS. The present invention more particularly relates to the use of the compound sildenafil, for the treatment of PCOS. A cyclic guanosine 3′,5′-monophosphate phosphodiesterase type five inhibitor is sometimes referred to as a cGMP PDE5 inhibitor or a cGMP PDE5i.

### POLYCYSTIC OVARY SYNDROME AND DIAGNOSIS

It is estimated that about 5% of pre-menopausal women suffer from PCOS. Women with PCOS are likely to experience problems with ovulation, and may have either a small amount of menses or no menses. Women with PCOS may experience hyperandrogenicity due to their increased levels of circulatory androgens and as such are likely to display symptoms of hirsuitism, virilisation and acne. The most common symptoms associated with PCOS are infertility, obesity, oligimenorrhoea and hirsuitism. Further symptoms frequently found in women with PCOS are amenorrhea, seborrhoea, acne, alopecia and impaired glucose tolerance. Rarer symptoms include hypertension, endometrial cancer and ovarian tumors.

The number of women with PCOS who have impaired glucose tolerance is estimated to be about 31%, whilst about 7.5% will have diabetes mellitus.

Biochemically PCOS in a subject can be indicated by: increased androgen levels, decreased sex-hormone binding globulin, increased LH/FSH ratio, acyclic oestrogen levels, hyperinsulinaemia, insulin resistance, increased PAI-1 levels. Symptoms commonly displayed by PCOS subjects who are insulin resistant include: obesity, diabetes mellitus and hypertension which are all cardiovascular risk factors.

Given the relatively high prevalence of insulin resistance in PCOS subjects current therapies commonly utilise insulin lowering/sensitising agents such as mefformin, troglitizone, PPAR-gamma.

Alternative therapies for various symptoms of PCOS include: clomid; oral contraceptives (oestrogens and progestins);GnRH analogues in combination with oral contraceptives; glurocorticoids to suppress adrenals; androgen receptor antagonists such as for example, spironolactone, cyproterone acetate, flutamide, 5-alpha reductase inhibitors or finasteride; andreogen biosynthesis inhibitors, such as for example ketoconazole; bromocriptine; cimetidine.

Infertility is a distressing condition for any woman and it is common for women with PCOS to experience difficulties in conceiving. Applicants have found that sildenafil demonstrates effects on key clinical parameters associated with both the development of PCOS and of PCOS itself.

It is therefore an object of the present invention to provide a treatment for PCOS via use of sildenafil or a pharmaceutically acceptable salt thereof.

It is a further aspect to provide a means for prevention of the development of PCOS via treatment with sildenafil or a pharmaceutically acceptable salt thereof. For example, a female presenting with a number of the risk factors for the development of PCOS, such as insulin resistance, hypertension, obesity for example could be treated with sildenafil as a preventative measure.

It is an additional object of the present invention to provide a treatment for infertility associated with PCOS via treatment with sildenafil or a pharmaceutically acceptable salt thereof.

A discussion of PCOS may be found in BMJ 1998, vol. 317, pages 329-332. Certain teachings from this article are now presented below:

“Polycystic ovarian syndrome is the most common form of anovulatory infertility. Its association with menstrual disturbance and altered hormonal parameters leads many affected women of reproductive age to attend a gynaecology or infertility clinic. The aetiology of the condition is unknown, but recent evidence suggests that the principal underlying disorder is one of insulin resistance, with the resultant hyperinsulinaemia stimulating excess ovarian androgen production. Associated with the prevalent insulin resistance, these women exhibit a characteristic dyslipidaemia and a predisposition to non-insulin dependent diabetes and cardiovascular disease in later life. Thus, polycystic ovarian syndrome seems to have many of the hallmarks of the metabolic syndrome.

Diagnostic clinical features of polycystic ovarian syndrome include menstrual disturbance, secondary to chronic anovulation or oligoovulation, and hirsutism or acne due to hyperandrogenaemia. Despite this classic concept, it is a heterogeneous disorder and exact diagnostic criteria remain contentious. Hence, along with racial variations, the prevalence of the condition can only be estimated at between 5% and 10% of women of reproductive age.

Elevated free testosterone activity, defined by the free androgen index, represents the most sensitive biochemical marker supporting the diagnosis. A raised luteinising hormone concentration, although a useful marker of the syndrome, is now less favoured as a diagnostic tool. Most, but not all, subjects show a characteristic ultrasound appearance of enlarged ovaries and an increased echo dense stroma surrounded by multiple, small, peripherally situated follicles. Exclusion of other possible aetiologies that may present in a similar fashion such as late onset congenital adrenal hyperplasia, thyroid disease, hyperprolactinaemia, and androgen secreting tumours is essential.

Good evidence supports the hypothesis that decreased peripheral insulin sensitivity and consequent hyperinsulinaemia are pivotal in the pathogenesis of polycystic ovarian syndrome. Peripheral insulin resistance is most evident in overweight patients: obesity and polycystic ovarian syndrome each seem to have a separate and synergistic relation with insulin resistance. The exact mechanism(s) for insulin resistance is uncertain, but a post-receptor defect in adipose tissue has been identified. Despite insulin resistance in adipose and skeletal muscle, the ovary remains relatively sensitive to insulin, and both insulin and insulin-like growth factor 1 have stimulatory effects on thecal androgen production. In fact, some lean women with polycystic ovarian syndrome, who may not have insulin resistance and therefore hyperinsulinaemia, may show enhanced ovarian sensitivity to insulin.

Insulin also acts on the liver to inhibit the production of sex hormone binding globulin and insulin-like growth factor 1 binding protein. A reduction in sex hormone binding globulin leads to an increase in the biologically available free testosterone. Thus, insulin resistance not only increases secretion of ovarian androgens but also promotes an increase in the proportion of free (active) hormone. Similarly, inhibition of production of insulin-like growth factor 1 binding protein results in an increased concentration of circulating free insulin-like growth factor 1, further enhancing ovarian androgen production.

Current consensus suggests that the ovary is the principal site of excess androgen production, but some women with polycystic ovarian syndrome may have an adrenal contribution to the increased androgen production. The mechanisms for this remain obscure and are almost certainly multifactorial. It is well recognised that visceral distribution of body fat, common in the syndrome, is of greater consequence to the metabolic effects of insulin resistance than obesity per se. Central obesity and insulin resistance lead to an altered lipolytic response to insulin, with impaired suppression of release of free fatty acids from adipose tissue. An increased flux of free fatty acids from central sites enters the portal circulation, increasing the availability of substrate to the liver for triglyceride production. Furthermore, women with the syndrome exhibit increased activity of hepatic lipase, an enzyme responsible for the conversion of large lipoprotein particles to smaller, more atherogenic species. This explains the findings of reduced concentrations of high density lipoprotein cholesterol and increased levels of atherogenic, small, low density lipoprotein. The combination of raised triglyceride and decreased high density lipoprotein is strongly linked with cardiovascular disease. Discrepancies in these lipid parameters, between patients with polycystic ovarian syndrome and controls matched for age and weight are evident at an early age. Hence, an increased risk of cardiovascular disease due to lipid perturbances will present in early adult life. Women with polycystic ovarian syndrome also show elevated concentrations of plasminogen activator inhibitor 1, a potent inhibitor of fibrinolysis, which have been shown to predict the occurrence of myocardial infarction.

Suppression of hyperandrogenaemia by use of gonadotrophin releasing hormone analogues has little effect on the insulin resistance or the dyslipidaemia, suggesting that the abnormal lipid profile is independent of the raised androgen concentrations.

Women with polycystic ovarian syndrome are currently treated according to their presenting features irregular menses, hirsutism, or infertility.

Irregular menses: The combined oral contraceptive pill is commonly used to regulate menses. By increasing levels of sex hormone binding globulin while decreasing androgen secretion, it reduces the circulating free testosterone activity. However, the combined pill exacerbates insulin resistance, and, since many patients are overweight and obesity is a relative contraindication, this treatment may be unsuitable.

Hirsutism: This may be addressed by the use of the antiandrogens cyproterone acetate or spironolactone (the former used in combination with ethinyloestradiol). Their principal mode of action is the inhibition of the binding of dihydrotestosterone to its receptor at the hair follicle. Beneficial effects can be seen after three months, but excessive hair growth returns soon after cessation of treatment. Cyproterone acetate may exacerbate irregularity of the menstrual cycle, and both drugs are unsuitable for use in those trying to conceive.

Infertility: For patients wishing to become pregnant, clomiphene citrate may be successful in stimulating ovulation but carries an increased risk of multiple pregnancy. By inhibiting the oestrogen mediated negative feedback loop at the hypothalamus, it enhances secretion of follicle stimulating hormone. Guidelines suggest that the duration of clomiphene treatment should not exceed six months because of the potential increased risk of ovarian cancer. Those failing to conceive after clomiphene treatment usually respond to exogenous gonadotrophins, but this requires intensive monitoring to reduce the risk of multiple conceptions.

Alternatives to medical treatment include laser or electrocautery of the ovary. This is often used as a last resort, is not available in all centres, and is difficult with obese patients. Although effective in aiding ovulation and regulating menses, its beneficial effects are usually short term.

Insulin resistance: As the principal underlying defect in polycystic ovarian syndrome seems to be insulin resistance, the most appropriate treatment for all clinical presentations may be one that specifically addresses this problem.

Weight reduction has multiple benefits for obese women with polycystic ovarian syndrome. The resultant reduction in insulin resistance corrects the hormonal imbalance, promotes ovulation and regular menses, and improves the metabolic consequences of the disorder. Weight loss should therefore be encouraged, but it seems to be hard to achieve for this group of patients.

Insulin sensitising agents: Recent trials have investigated the effect of such agents on polycystic ovarian syndrome. Mefformin, a biguanide often used in non-insulin dependent diabetes, has been the most commonly used. Troglitazone, a thiazolidinedione that improves muscle insulin sensitivity, has also been studied but has recently been removed from the market because of adverse effects on hepatic function. Trials to date have included only small numbers of subjects, but results have been promising, with most showing reductions in concentrations of fasting serum insulin, androgen, and luteinising hormone. In addition, circulating concentrations of sex hormone binding globulin increased, resulting in less bioactively available testosterone. Preliminary evidence indicates that treatment of obese women with polycystic ovarian syndrome with mefformin restores regular menstrual cycles and ovulation. Whether insulin sensitising agents can modify the vascular risk factors associated with the syndrome remains to be seen, but reductions in Lp (a) lipoprotein and plasminogen activator inhibitor 1 have been observed. Additionally, some studies have reported that treated subjects have shown some weight loss despite continuation of their normal diet and lifestyle, and others have demonstrated a reduction in central obesity.

Thus, treatments targeting the key factor in the disorder may not only resolve the gynaecological problems with which the syndrome presents, but also reduce the risk of vascular disease in later life.”

A discussion of PCOS may be found at http://www.mc.vanderbilt.edu/peds/pidl/adolesc/poIcysov.htm. For the ease of reference those teachings are now presented below:

“Polycystic ovarian syndrome (PCOS) was originally described in 1905 by Stein and Leventhal as a syndrome consisting of amenorrhea, hirsutism, and obesity in association with enlarged polycystic ovaries. It is now realized that this relatively common syndrome is an extremely heterogenous clinical syndrome that begins soon after menarche and some authors prefer to refer to it as a syndrome of hyperandrogenic chronic anovulation. In fact, earlier studies of PCOS have focused on ovarian morphological findings and were considered to be an important diagnostic criteria. However, it was found that polycystic changes of the ovaries were observed in some nomially cycling women.

Furthermore, polycystic changes of the ovaries were shown to be associated with other well-defined diseases such as Cushing's syndrome, and an ovarian or adrenal tumor capable of producing androgen.

In addition, recent studies have demonstrated that some women with characteristic clinical features of PCOS have normal-sized ovaries. Indeed, nothing inherently abnormal has thus far been found in the ovaries of PCOS. Therefore, the focus on ovarian morphology was shifted towards the hormonal characteristics of the syndrome. The incidence of PCOS is about 3% in both adolescents and adults. It is the most common cause of hyperandrogenisim of prepubertal onset. However, it appears that there is some variabilities of PCOS clinical manifestations among races. Obesity and hirsutism are not pronounced in Japanese women with PCOS. In the United States, 70% of patients have hirsutism compared to 10-20% in Japan and the Orient. Obesity, although thought to be common in PCOS is usually noted in 40% of cases. There is no particular pattern with respect to fat distribution. However, obesity is an important feature with regard to hirsutism because it is associated with decreased sex hormone binding globulin (SHBG), which results in an increased fraction of unbound testosterone.

In addition, obesity contributes to chronic estrogen stimulation because there is increased peripheral conversion of androgen to estrogens in these patients. Among women with resistant acne, not responding to conventional treatments, the polycystic ovary syndrome is very common. The primary affected areas are the facial (angle of the jaw, upper lip, and chin) and suprapubic region of the body. Other common sites include the chest, inner thigh, and perineum. Another clinical sign in hyperandrogenic syndromes is acanthosis nigricans. It is characterized by symmetric, velvet-like, grey-brown hyperpigmentation of the skin. It commonly affects the nape of the neck, axillae and groin.

The most common features of PCOS are chronic anovulation and infertility in addition to the hyperandrogenism. The clinical manifestation of chronic anovulation include irregular menstrual cycles, oligo or amenorrhea interspersed with heavy vaginal bleeding. The menstrual dysfunction usually presents from menarche. In the absence of ovulation, the usual premenstrual molimina does not occur. In addition, because there is unopposed estrogen stimulation of the endometrium, endometrial hyperplasia and in some instances, adenocarcinoma may develop.

Fortunately, adrenocarcinomas associated with PCOS is usually of low histologic grade and presents at an early stage. In PCOS, chronic anovulation reflects abnormal folliculogenesis. As a result, these patients suffer from infertility. Occasionally, spontaneous ovulation and pregnancy may occur in this syndrome. A family history may be present in a subset of patients. However, so far, efforts to elucidate a particular mode of genetic inheritance have been unsuccessful. PCOS is an endocrinologic disorder of undetermined etiology characterized by inappropriate gonadotropinreleasing hormone (GnRH) pulse amplitude and tonically elevated levels of luteinizing hormone (LH), but not of follicle-stimulating hormone (FSH).

In addition, there are increased circulatory levels of androgens produced by both the ovaries and the adrenal glands. If they are elevated, serum testosterone levels are usually between 70-120 ng/dl, and androstenedione levels are usually between 3 and 5 ng/ml. Also, about half the women with this syndrome have elevated DHEA-S. The presence or absence of hirsutism depends on whether these androgens are converted peripherally by 5 alpha reductase to the more potent androgen DHT dihydrotestosterone and 3 alpha diol-G as reflected by increased levels of 3 alpha-diol-G. Therefore, it is skin 5-alpha reductase activity that largely determine the presence or absence of hirsutism. The chronically elevated LH are usually above 20 mlU/ml. Because FSH levels in PCOS patients are normal or low, it has been found that an LH/FSH ratio greater than 3, provided the LH level is not lower than 8mlU/ml, may be used to suggest the diagnosis in women with clinical features of PCOS. About 20% of women with PCOS also have mildly elevated levels of prolactin (20-30 ng/ml), possibly related to increased pulsatility of GnRH or to a relative dopamine deficiency or to both. In addition, many women with this syndrome have mild degrees of hyperinsulinism and insulin resistance.

The diagnosis of PCOS is strongly suggested by the clinical history and physical examination. In particular, a pattern of infrequent and irregular menstruation commencing at time of puberty is highly suggestive. Evidence of concomitant excessive hair is almost pathognomonic. The most worrisome consideration in the hirsute woman is the presence of an androgen-producing neoplasm. It is for this reason that a measurement of total testosterone and DHEA-S is recommended. A level greater than 200 ng/dl, as determined by radioimmunoassay with chromatographic separation should raise suspicion of an androgen-producing tumor of ovarian or adrenal origin. Serum DHEA-S is the marker of adrenal androgen and a level greater than 700 ng/dl implies a possible neoplasm. Mild to moderate hirsutism may reflect the presence of CAH, 21 hydroxylase deficiency, although severe hirsutism is frequently the case.

Other characteristic clinical findings associated with hirsutism in this disorder include regular menstrual cycles, virilization such as clitoromegaly, family history, and short stature. Although 17-OH progesterone is elevated in both PCOS and CAH, 21 hydroxylase deficiency, levels rarely exceed 300 ng/dl in PCOS. Therefore, concentration above 300 ng/dl suggest CAH, 21 hydroxylase deficiency and ACTH stimulation should be performed. Other enzyme defects of CAH that give rise to hirsutism are deficiencies of 11-beta hydroxylase deficiency and 3-beta hydroxylase deficiency. Diagnosis of the former is suggested by the presence of coexistent hypertension and salt retention, whereas the latter condition is associated with a marked elevation of serum DHEA-S.”

### SUMMARY ASPECTS OF THE INVENTION

In accordance with the present invention we have surprisingly found that it is possible to treat PCOS with the use of a PDE5 inhibitor.

The PDE5 inhibitor may be used in combination with one or more additional pharmaceutically active agents (for simultaneous, separate or sequential administration). The additional pharmaceutically active agent(s), if either present or used in conjunction with the PDE5 inhibitor of the present invention, may be referred to as an “additional agent” or “additional active agent”.

The additional agent, may, for example, be one or more other agents useful in the treatment of PCOS.

Such combinations of PDE5 inhibitors and additional agents are discussed in more detail below.

Thus the present invention additionally comprises the combination of a PDE5 inhibitor for the treatment of PCOS (as detailed herein) with one or more additional agents.

### DETAILED ASPECTS OF THE PRESENT INVENTION

According to a first aspect, the present invention provides the use of a PDE5 inhibitor in the preparation of a medicament for the treatment of PCOS.

According to a second aspect, the present invention provides the use of a pyrazolopyrimidinone PDE5 inhibitor in the preparation of a medicament for the treatment of PCOS.

According to a third and preferred aspect, the present invention provides the use of the compound sildenafil or pharmaceutically acceptable salts thereof in the preparation of a medicament for the treatment of PCOS.

According to a fourth aspect, the present invention provides a method of treating PCOS in an individual which comprises administering to said individual an effective amount of sildenafil or a pharmaceutically acceptable salt thereof.

According to a fifth aspect, the present invention provides a pharmaceutical composition for use in the treatment of PCOS comprising sildenafil or a pharmaceutically acceptable salt thereof admixed with a pharmaceutically acceptable carrier, diluent or excipient.

In the above defined fifth aspect of the present invention, the pharmaceutical composition may additionally comprise one or more additional active agents.

According to a sixth aspect, the present invention provides a pharmaceutical combination (for simultaneous, separate or sequential administration) for the treatment of PCOS in an individual comprising sildenafil or a pharmaceutically acceptable salt thereof and one or more additional active agents.

According to a seventh aspect, the present invention provides a method of preparing a pharmaceutical composition for use in the treatment of PCOS comprising admixing sildenafil or a pharmaceutically acceptable salt thereof with a pharmaceutically acceptable carrier, diluent or excipient.

For ease of reference, these and further aspects of the present invention are now discussed under appropriate section headings. However, the teachings under each section are not necessarily limited to each particular section.

As used herein, the terms “pharmaceutical” and “pharmaceutically” may include “veterinary” and “veterinarily”, respectively.

### INDIVIDUAL

As used herein, the term “individual” refers to female vertebrates, particularly female members of the mammalian species.

### TREATMENT

It is to be understood that all references herein to treatment include one or more of curative, palliative and prophylactic treatment. Preferably, the term treatment includes at least curative treatment and/or palliative treatment.

Further, it is to be appreciated that all references herein to treatment include acute treatment (taken as required) and chronic treatment (longer term continuous treatment).

### INHIBITOR

The term “inhibitor” as used herein with respect to the agent of the present invention means an agent that can reduce and/or eliminate and/or mask and/or prevent the detrimental action of PDE5. The inhibitor may act as an antagonist.

### PDE5 INHIBITOR

The term PDE5 inhibitor includes the inhibitor per se and/or a pharmaceutically acceptable salt, solvate or composition thereof.

The PDE5 inhibitors used in the present invention are sometimes referred to herein as cyclic guanosine 3′,5′-monophosphate phosphodiesterase type five inhibitors or cGMP PDE5 inhibitors or an agent (that is an agent according to the present invention).

Preferably suitable pyrazolopyrimidinone PDE5 inhibitors for use in accordance with the present invention are compounds which are a selective inhibitor of the PDE5 isoenzyme.

For more preferable aspects of the present invention the PDE5 inhibitor is a compound which is a highly selective inhibitor of the PDE5 isoenzyme.

The suitability of any particular PDE5 inhibitor can be readily determined by evaluation of its potency and selectivity using literature methods followed by evaluation of its toxicity, absorption, metabolism, pharmacokinetics, etc in accordance with standard pharmaceutical practice.

IC50 values for the PDE5 inhibitors may be determined using the PDE5 assay in the Assay section hereinafter. Preferably, the PDE5 inhibitors have an IC50 against the PDE5 enzyme of less than 100 nanomolar (more preferably, at less than 50 nanomolar).

As stated hereinbefore, preferably the PDE5 inhibitors used according to the present invention are selective for the PDE5 enzyme. Preferably (e.g. when used orally) they are selective over PDE3, more preferably over PDE3 and PDE4. Preferably (e.g. when oral), the PDE5 inhibitors of the invention have a selectivity ratio greater than 100 more preferably greater than 300, over PDE3 and more preferably over PDE3 and PDE4. Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

More preferably the preferred PDE5 compounds of the present invention are pyrazolopyrimidinones which are highly selective for PDE5 and display desirable selectivity for PDE5 versus PDE6. Especially preferred herein are sildenafil, sildenafil citrate and sildenafil mesylate.

For some applications, preferably the PDE5 inhibitor of the present invention (and optionally the optional additional agent) has a Kᵢ value of less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the PDE5 inhibitor of the present invention (and optionally the optional additional agent) has a Kb value of less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the PDE5 inhibitor of the present invention (and optionally the optional additional agent) has a Ka value of less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

Especially preferred herein is the combination of one or more potent and selective cGMP PDE5 inhibitors with one or more selective D3 dopamine receptor agonists.

### PARTICULAR PDE5 COMPOUNDS

Particularly useful compounds for use in the present invention have the general formula (I):

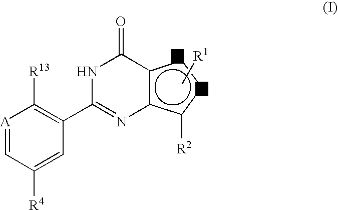

wherein:

A is CH or N;

R¹ is H, C₁ to C₆ alkyl, C₃ to C₆ alkenyl, C₃ to C₆ cycloalkyl, C₃ to C₆ cycloalkenyl, or C₁-C₃ perfluoroalkyl, wherein said alkyl group may be branched or straight chain and wherein said alkyl, alkenyl, cycloalkyl or perfluoroalkyl group is optionally substituted by; one or more substituents selected from: hydroxy; C₁ to C₄ alkoxy; C₃ to C₆ cycloalkyl; C₁-C₃ perfluoroalkyl; phenyl substituted with one or more substitutents selected from C₁ to C₃ alkyl, C₁ to C₄ alkoxy, C₁ to C₄ haloalkyl or C₁ to C₄ haloalkoxy wherein said haloalkyl and haloalkoxy groups contain one or more halo atoms, halo, CN, NO₂, NHR¹¹, NHSO₂R¹², SO₂R¹², SO₂NHR¹¹, COR¹¹, CO₂R¹¹ wherein R¹¹ is H, C₁ to C₄ alkyl, C₂ to C₄ alkenyl, C₁ to C₄ alkanoyl, C₁ to C₄ haloalkyl or C₁ to C₄ haloalkoxy and wherein R¹² is C₁ to C₄ alkyl, C₂ to C₄ alkenyl, C, to C₄ alkanoyl, C, to C₄ haloalkyl or C, to C₄ haloalkoxy; NR⁷R⁸, CONR⁷R⁸ or NR⁷COR¹¹ wherein R⁷ and R⁸ are each independently selected from H, C₁ to C₄ alkyl, C₂ to C₄ alkenyl, C₁ to C₄ alkoxy, CO₂R⁹, SO₂R⁹ wherein said alkyl, alkenyl or alkoxy groups are optionally substituted by NR⁵R⁶, C₁ to C₄ haloalkyl or C₁ to C₄ haloalkoxy and wherein R⁹ is H, hydroxy C₂ to C₃ alkyl, C₁ to C₄ alkanoyl or C₁ to C₄ alkyl which is optionally substituted with phenyl wherein said phenyl group is optionally substituted by one or more substituents selected from C₁ to C₄ alkyl optionally substituted by C₁ to C₄ haloalkyl or C₁ to C₄ haloalkoxy, C₁ to C₄ alkoxy, halo, CN, NO₂, NHR¹¹, NHSO₂R¹², SO₂R¹², SO₂NHR¹¹, COR¹¹ or CO₂R″; Het¹; Het² or Het³; or R¹ is Het⁴ or phenyl wherein said phenyl group is optionally substituted by one or more substituents selected from C₁ to C₄ alkyl, C₂ to C₄ alkenyl, C₁ to C₄ alkoxy, halo, CN, CF₃, OCF₃, NO₂, NHR¹¹, NHSO₂R¹², SO₂R¹², SO₂NHR¹¹, COR¹¹, CO₂R¹¹;

R² is H, C₁ to C₆ alkyl, C₃ to C₆ alkenyl or (CH₂)ₙ(C₃ to C₆ cycloalkyl) wherein n is 0, 1 or 2 and wherein said alkyl or alkyenyl group is optionally substituted with one or more fluoro substituents;

R¹³ is OR³ or NR⁵R⁶;

R³ is C₁ to C₆ alkyl, C₃-C₆ alkenyl, C₃-C₆ alkynyl, C₃-C₇ cycloalkyl, C₁-C₆ perfluoroalkyl or (C₃-C₆ cycloalkyl)C₁-C₆ alkyl optionally substituted with one or two substituents selected from C₃ to C₅ cycloalkyl, hydroxy, C₁ to C₄ alkoxy, C₃-C₆ alkenyl, C₃-C₆ alkynyl, benzyloxy, NR⁵R⁶, phenyl, Het¹, Het², Het³ or Het⁴ wherein the C₁ to C₆ alkyl and C₁ to C₄ alkoxy groups may optionally be terminated by a haloalkyl group such as CF₃; C₃ to C₆ cycloalkyl; Het¹, Het², Het³ or Het⁴;

R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; hydroxy C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; or phenyl or heterocyclyl either of which is optionally substituted with methyl; or R⁴ is a pyrrolidinylsulphonyl, piperidinosulphonyl, morpholinosulphonyl, or piperazin-1-ylsulphonyl group having a substituent, R¹⁰ at the 4-position of the piperazinyl group wherein said piperazinyl group is optionally substituted with one or two C₁ to C₄ alkyl, C₁ to C₃ alkoxy, NR⁷R⁸ or CON R⁷R⁸ groups and is optionally in the form of its 4-N-oxide;

R⁵ and R⁶ are each independently selected from H and C₁ to C₄ alkyl optionally substituted with C₃ to C₅ cycloalkyl or C₁ to C₄ alkoxy, or, together with the nitrogen atom to which they are attached, form an azetidinyl, pyrrolidinyl, piperidinyl, morpholinyl, 4-(NR⁹)- piperazinyl or imidazolyl group wherein said group is optionally substituted with methyl or hydroxy;

R¹⁰ is H; C₁ to C₆ alkyl, (C₁-C₃ alkoxy) C₂-C₆ alkyl, hydroxy C₂-C₆ alkyl, (R⁷R⁸N)C₂-C₆ alkyl, (R⁷R⁸NCO)C₁-C₆ alkyl, CONR⁷R⁸, CSNR⁷R⁸ or C(NH)NR⁷R⁸ optionally substituted with one or two substituents selected from hydroxy, NR⁵R⁶, CONR⁵R⁶, phenyl optionally substituted with C₁ to C₄ alkyl or C₁ to C₄ alkoxy; C₂ to C₆ alkenyl or Het⁴;

Het¹ is an N-linked 4-, 5- or 6-membered nitrogen-containing heterocyclic group optionally containing one or more further heteroatoms selected from S, N or O;

Het² is a C-linked 5-membered heterocyclic group containing an O, S or N heteroatom optionally containing one or more heteroatoms selected from O or S;

Het³ is a C-linked 6-membered heterocyclic group containing an O or S heteroatom optionally containing one or more heteroatoms selected from O, S or N or Het³ is a C-linked 6-membered heterocyclic group containing three N heteroatoms;

Het⁴ is a C-linked 4-, 5- or 6-membered heterocyclic group containing one, two or three heteroatoms selected from S, O or N; and wherein any of said heterocyclic groups Het¹, Het², Het³ or Het⁴ may be saturated, partially unsaturated or aromatic and wherein any of said heterocyclic groups may be optionally substituted with one or more substituents selected from C₁ to C₄ alkyl, C₂ to C₄ alkenyl, C₁ to C₄ alkoxy, halo, CO₂R¹¹, COR¹¹, SO₂R¹² or NHR¹¹ and/or wherein any of said heterocyclic groups is benzo-fused.

or wherein when R¹³ represents OR³ or R³NR⁵; R¹ represents Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R² represents H, halo, cyano, nitro, OR , OC(O)R⁷, C(O)R⁸, C(O)OR⁹, C(O)NR¹⁰R¹¹, NR¹²R¹³, SO₂NR¹⁴R¹⁵, lower alkyl, Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R³ represents H, lower alkyl, alkylHet or alkylaryl, which latter three groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R⁴ represents H, halo, cyano, nitro, halo(loweralkyl), OR⁶, OC(O)R⁷, C(O)R⁸, C(O)OR⁹, C(O)NR¹⁰R¹¹, NR¹²R¹³, NR¹⁶Y(O)R¹⁷, SOR¹⁸, SO₂R¹⁹R²⁰, C(O)AZ, lower alkyl, lower alkenyl, lower alkynyl, Het, alkylHet, aryl, alkylaryl, which latter seven groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; Y represents C or S(O), wherein one of R¹⁶ and R¹⁷ is not present when Y is S(O); A represents lower alkylene; Z represents OR⁶, halo, Het or aryl, which latter two groups are both optionally substituted with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R⁵, R⁶, R⁷, R⁸, R⁹, R¹⁸, R¹⁹ and R²⁰ independently represent H or lower alkyl; R¹⁰ and R¹¹ independently represent H or lower alkyl, which latter group is optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵ or Het or aryl optionally substituted with one or more of said latter eleven groups or one of R¹⁰ and R¹¹ may be lower alkoxy, amino or Het, which latter two groups are both optionally substituted with lower alkyl; R¹² and R¹³ independently represent H or lower alkyl or one of R¹² or R¹³ may be C(O)—lower alkyl or C(O)Het in which Het is optionally substituted with lower alkyl; R¹⁴ and R¹⁵ independently represent H or lower alkyl or R¹⁴ and R¹⁵, together with the nitrogen atom to which they are bound, form a heterocyclic ring; R¹⁶ and R¹⁷ independently represent H or lower alkyl or one of R¹⁶ and R¹⁷ may be Het or aryl, which latter two groups are both optionally substituted with lower alkyl; Het represents an optionally substituted four to twelve membered heterocyclic group, which may be aromatic or non-aromatic, which may contain one or more double bonds, which may be mono- or bi-cyclic and which contains one or more heteroatoms selected from N, S and O;

or a pharmaceutically acceptable salt or solvate of any thereof.

The PDE5 inhibitor may contain halo groups. Here, “halo” means fluoro, chloro, bromo or iodo.

The PDE5 inhibitor may contain one or more of alkyl, alkoxy, alkenyl, alkylene and alkenylene groups—which may be unbranched- or branched-chain.

A preferred group of compounds of general formula (I) for use according to the present invention are those wherein: R¹ is H, methyl or ethyl; R² is H, C₁-C₃ alkyl optionally substituted by OH, or methoxy; R³ is C₂-C₃ alkyl or allyl; R⁴ is a sulphonylpiperidino or 4-N-(R¹⁰)-sulphonylpiperazin-1-yl group; R⁵ is H, NR⁷R⁸, or CONR⁷R⁸; R¹⁰ is H, C₁-C₃ alkyl, hydroxy C₂-C₆ alkyl, CONR⁷R⁸, CSNR⁷R⁸ or C(NH)NR⁷R⁸; R⁷ and R⁸ are each independently H or methyl.

Another preferred group of compounds of general formula (I) for use according to the present invention are those wherein: R¹ is C₁ to C₂ alkyl optionally substituted with Het; 2-(morpholin-4-yl)ethyl or benzyl; R² is C₂ to C₄ alkyl; R¹³ is OR³ or NR⁵R⁶; R³ is C₁ to C₄ alkyl optionally substituted with one or two substituents selected from cyclopropyl, cyclobutyl, OH, methoxy, ethoxy, benzyloxy, NR⁵R⁶, phenyl, furan-3-yl, pyridin-2-yl and pyridin-3-yl; cyclobutyl; 1-methylpiperidin-4-yl; tetrahydrofuran-3-yl or tetrahydropyran-4-yl; R⁵ and R⁶ are each independently selected from H and C₁ to C₂ alkyl optionally substituted with cyclopropyl or methoxy, or, together with the nitrogen atom to which they are attached, form a azetidinyl, pyrrolidinyl or morpholinyl group; R⁷ and R⁸, together with the nitrogen atom to which they are attached, form a 4-R¹⁰-piperazinyl group optionally substituted with one or two methyl groups and optionally in the form of its 4-N-oxide; R¹⁰ is H, C₁ to C₃ alkyl optionally substituted with one or two substituents selected from OH, NR⁵R⁶, CONR⁵R⁶, phenyl optionally substituted with methoxy, benzodioxol-5-yl and benzodioxan2-yl; allyl; pyridin-2-yl; pyridin-4-yl or pyrimidin-2-yl; and Het is selected from pyridin-2-yl; 1-oxidopyridin-2-yl; 6-methylpyridin-2-yl; 6-methoxypyridin-2-yl; pyridazin-3-yl; pyrimidin-2-yl and 1-methylimidazol-2-yl. Of this group more preferred are those compounds wherein R¹ is C₁ to C₂ alkyl optionally substituted with Het; 2-(morpholin-4-yl)ethyl or benzyl; R² is C₂ to C₄ alkyl; R¹³ is OR³; R³ is C₁ to C₄ alkyl optionally monosubstituted with cyclopropyl, cyclobutyl, OH, methoxy, ethoxy, phenyl, furan-3-yl or pyridin-2-yl; cyclobutyl; tetrahydrofuran-3-yl or tetrahydropyran-4-yl; R⁷ and R⁸, together with the nitrogen atom to which they are attached, form a 4-R¹⁰-piperazinyl group optionally in the form of its 4-N-oxide; R¹⁰ is C₁ to C₃ alkyl optionally monosubstituted with OH; and Het is selected from pyridin-2-yl; 1oxidopyridin-2-yl; 6-methylpyridin-2-yl; 6-methoxypyridin-2-yl; pyridazin-3-yl; pyrimidin-2-yl and 1-methylimidazol-2-yl.

One other further preferred group of compounds of general formula (I) for use according to the present invention are those wherein: R¹ is C₁ to C₆ alkyl or C₃ to C₆ alkenyl wherein said alkyl or alkenyl groups may be branched chain or straight chain or R¹ is C₃ to C₆ cycloalkyl or C₄ to C₆ cycloalkenyl and wherein when R¹ is C₁ to C₃ alkyl said alkyl group is substituted by; and wherein when R¹ is C₄ to C₆ alkyl, C₃ to C₆ alkenyl, C₃ to C₆ cycloalkyl or C₄ to C₆ cycloalkenyl said alkyl, alkenyl, cycloalkyl or cycloalkenyl group is optionally substituted by; one or more substituents selected from: hydroxy; C₁ to C₄ alkoxy; C₃ to C₄ cycloalkyl; phenyl substituted with one or more substitutents selected from C₁ to C₃ alkyl, C₁ to C₄ alkoxy, C₁ to C₄ haloalkyl or C₁ to C₄ haloalkoxy, halo, CN, NO₂, NHR¹¹, NHCOR¹², NHSO₂R¹², SO₂R¹², SO₂NHR¹¹, COR¹¹, CO₂R¹¹ wherein said haloalkyl and haloalkoxy groups contain one or more halo atoms; NR⁷R⁸, CONR⁷R⁸ or NR⁷COR¹¹; a Het¹ group which is an N-linked 4-membered N-containing heterocyclic group; a Het² group which is a C-linked 5-membered heterocyclic group containing an O, S or N heteroatom optionally containing one or more heteroatoms selected from N, O or S; a Het³ group which is a C-linked 6-membered heterocyclic group containing an O or S heteroatom optionally containing one or more heteroatoms selected from O, S or N or a Het³ group which is a C-linked 6-membered heterocyclic group containing three N heteroatoms; wherein R⁷, R⁸, R¹¹ and R¹² are as previously defined herein or R¹ is a Het⁴ group which is a C-linked 4- or 5-membered heterocyclic group containing one heteroatom selected from S, O or N; a Het⁴ group which is a C-linked 6-membered heterocyclic group containing one, two or three heteroatoms selected from S or O; a Het⁴ group which is a C-linked 6-membered heterocyclic group containing three nitrogen heteroatoms; a Het⁴ group which is a C-linked 6-membered heterocyclic group containing one or two nitrogen heteroatoms which is substituted by one or more substitutents selected from C₁ to C₄ alkyl, C₁ to C₄ alkoxy, CO₂R¹¹, SO₂R¹², COR¹¹, NHR¹¹ or NHCOR¹² and optionally including a further heteroatom selected from S, O or N wherein any of said heterocyclic groups Het¹, Het², Het³ or Het⁴ is saturated, partially unsaturated or aromatic as appropriate and wherein any of said heterocyclic groups is optionally substituted with one or more substituents selected from C₁ to C₄ alkyl, C₃ to C₄ alkenyl, C₁ to C₄ alkoxy, halo, CO₂R¹¹, SO₂R¹², COR¹¹ or NHR¹¹ wherein R¹¹ is as defined hereinbefore and/or wherein any of said heterocyclic groups is benzo-fused; or R¹ is phenyl substituted by one or more substituents selected from CF₃, OCF₃, SO₂R¹² or CO₂R¹² wherein R¹² is C₁ to C₄ alkyl which is optionally substituted by phenyl, C₁ to C₄ haloalkyl or C₁ to C₄ haloalkoxy wherein said haloalkyl and haloalkoxy groups contain one or more halo atoms; R² is C₁ to C₆ alkyl; R¹³ is OR³; R³ is C₁ to C₆ alkyl optionally substituted with one or two substituents selected from C₃ to C₅ cycloalkyl, hydroxy, C₁ to C₄ alkoxy, benzyloxy, NR⁵R⁶, phenyl, furanyl, tetrahydrofuranyl or pyridinyl wherein said C₁ to C₆ alkyl and C₁ to C₄ alkoxy groups may optionally be terminated by a haloalkyl group such as CF₃; or R³ is C₃ to C₆ cycloalkyl, 1-(C₁ to C₄ alkyl)piperidinyl, tetrahydrofuranyl or tetrahydropyranyl; R⁴ is a piperazin-1-ylsulphonyl group having a substituent R¹⁰ at the 4-position of the piperazinyl group wherein said piperazinyl group is optionally substituted with one or two C₁ to C₄ alkyl groups and is optionally in the form of its 4-N-oxide; R⁵ and R⁶ are each independently selected from H and C₁ to C₄ alkyl optionally substituted with C₃ to C₅ cycloalkyl or C₁ to C₄ alkoxy, or, together with the nitrogen atom to which they are attached, form an azetidinyl, pyrrolidinyl, piperidinyl or morpholinyl group; and R¹⁰ is H; C₁ to C₄ alkyl optionally substituted with one or two substituents selected from hydroxy, NR⁵R⁶, CONR⁵R⁶, phenyl optionally substituted with C₁ to C₄ alkyl or C₁ to C₄ alkoxy; C₃ to C₆ alkenyl; Het⁴; with the proviso that when R¹ is C₁ to C₃ alkyl substituted by phenyl then said phenyl group is not substituted by C₁ to C₄ alkoxy; CN; halo; CF₃; OCF₃; or C₁ to C₄ alkyl. More preferred of this group of compounds are those wherein R¹ is C₁ to C₆ alkyl wherein said alkyl may be branched or straight chain or R¹ is C₃ to C₆ cycloalkyl and wherein when R¹ is C₁ to C₃ alkyl said alkyl group is substituted by; and wherein when R¹ is C₄ to C₆ alkyl or C₃ to C₆ cycloalkyl said alkyl or cycloalkyl group is optionally substituted by; one or more substituents selected from: hydroxy; C₁ to C₂ alkoxy; C₃ to C₅ cycloalkyl; NR⁷R⁸, NR⁷COR¹¹ or COR¹¹ wherein R⁷ and R⁸ are each independently selected from H, C₁ to C₄ alkyl or CO₂R⁹ wherein R⁹ and R¹¹ are as previously defined herein; a Het¹ group which is an N-linked 4-membered N-containing heterocyclic group; a Het³ group which is a C-linked 6-membered heterocyclic group containing an O or S heteroatom optionally containing one or more heteroatoms selected from O, S or N or a Het³ group which is a C-linked 6-membered heterocyclic group containing three N heteroatoms; or R¹ is a Het⁴ group which is a C-linked 4-membered heterocyclic group containing one heteroatom selected from S, O or N or R¹ is a Het⁴ group which is a C-linked 6-membered heterocyclic group containing one, two or three heteroatoms selected from S or O wherein any of said heterocyclic groups Het¹, Het², Het³ or Het⁴ is saturated, partially unsaturated or aromatic and is optionally substituted with one or more substituents selected from C₁ to C₄ alkyl, C₁ to C₄ alkoxy, —CO₂R¹¹, —SO₂R¹², —COR¹¹ or NHR¹¹ wherein R¹¹ and R¹² are as defined hereinbefore and/or wherein any of said heterocyclic groups is benzo-fused; or R¹ is phenyl substituted by one or more substituents selected from : CF₃, —OCF₃, —SO₂R¹², —COR, —CO₂R¹¹ wherein R¹¹ and R¹² are as defined hereinbefore; R² is C₁ to C₆ alkyl; R¹³ is OR³; R³ is methyl, ethyl, n-propyl, i-propyl, n-butyl, sec-butyl, i-butyl or t-butyl alkyl optionally substituted with one or two substituents selected from cyclopropyl, cyclobutyl, hydroxy, methoxy, ethoxy, benzyloxy, phenyl, benzyl, furan-3-yl, tetrahyd rofuran-2-ylmethyl, tetrahyd rofuran-3-ylmethyl, pyridin-2-yl, pyridin-3-yl or NR⁵R⁶ wherein R⁵ and R⁶ are each independently selected from H and C, to C₂ alkyl; R⁴ is a piperazin-1-ylsulphonyl group having a substituent, R¹⁰ at the 4-position of the piperazinyl group wherein said piperazinyl group is optionally substituted with one or two C₁ to C₄ alkyl groups and is optionally in the form of its 4-N-oxide; and R¹⁰ is H, C₁ to C₃ alkyl optionally substituted with one or two substituents selected from hydroxy, NR⁵R⁶, CONR⁵R⁶ wherein R⁵ and R⁶ are each independently selected from H, C₁ to C₄ alkyl and C₃ alkenyl.

A further group of preferred compounds of general formula (I) for use according to the present invention are those wherein: R¹ represents H, lower alkyl, Het, alkylHet, or alkylaryl (which latter four groups are all optionally substituted and/or terminated with one or more substituents selected from cyano, lower alkyl, OR⁶, C(O)OR⁹ or NR¹²R¹³); R² represents H, halo, lower alkyl, Het or aryl (which latter three groups are all optionally substituted and/or terminated with one or more substituents as defined hereinbefore, and preferably with NR¹²R¹³ or SO₂NR¹⁴R¹⁵); R³ represents C₁-C₄ alkyl or C₃-C₄ cycloalkyl which are optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵); R⁴ represents halo, cyano, nitro, C(O)R⁸, C(O)OR⁹, C(O)NR¹⁰R¹¹, NR¹²R¹³, N[Y(O)R¹⁷]₂, NR¹⁶Y(O)R¹⁷, SOR¹⁸, SO₂R¹⁹, C(O)AZ, lower alkyl, lower alkynyl, Het or aryl, which latter three groups are all optionally substituted and/or terminated with one or more substituents as defined hereinbefore; and wherein Y, A, Z, R¹⁰, R¹¹, R¹², R¹³, R¹⁴, R¹⁵, R¹⁶, R¹⁷, R⁵ R⁶, R⁷, R⁸, R⁹, R¹⁸, R¹⁹, and Het are as herein before defined. More preferred in this further group are compounds in which R¹ represents optionally substituted lower alkyl, more preferably lower alkyl, lower alkoxy-terminated lower alkyl, NR¹²R¹³-terminated lower alkyl, or N-morpholino-terminated lower alkyl. Alternatively, R¹ may represent a 4-piperidinyl or a 3-azetidinyl group, optionally substituted at the nitrogen atom of the piperidinyl group with lower alkyl or C(O)OR⁹. In such more preferred compounds in this further group R² represents C(O)NR¹⁰R¹¹, NR¹²R¹³, lower alkyl optionally interrupted by one or more of O, S or N, optionally substituted at N by lower alkyl or acyl, or optionally substituted aryl or Het. More preferably, when R² is interrupted lower alkyl, the interrupting atoms are one or more of O and lower alkylated-N and when R² is aryl, it is optionally substituted phenyl or pyridyl. Particularly preferred compounds of this further group are those in which R² represents C(O)NR¹⁰R¹¹, NR¹²R¹³, C₁₄ alkyl optionally interrupted by O or N, optionally substituted at N by lower alkyl, optionally substituted phenyl, or optionally substituted pyridin-2-yl, pyridin-3-yl, pyrimidin-5-yl, pyrazin-2-yl, pyrazol-4-yl, oxadiazol-2-yl, furan-2-yl, furan-3-yl, tetrahydrofuran-2-yl and imidazo[1,2-a]pyridin-6-yl. In this more preferred group of further compounds R³ may represent lower alkyl or cycloalkyl. Also, X is preferably O. Such further and more preferred compounds have R⁴ representing halo, lower alkyl, lower alkynyl, optionally substituted Het, optionally substituted aryl, C(O)R⁸, C(O)AZ, C(O)OR⁹, C(O)NR¹⁰R¹¹, NR¹²R¹³ or NR¹⁶Y(O)R¹⁷. More preferred values for R⁴ are C(O)R⁸ (e.g. acetyl), halo (e.g. iodo), SO₂R¹⁹ (wherein R¹⁹ represents lower alkyl) and C(O)NR¹⁰R¹¹ (e.g. where R¹⁰ and R¹¹ independently represent H and lower alkyl and/or one of R¹⁰ and R¹¹ is lower alkoxy) or NHB, wherein B represents H, SO₂CH₃ or C(O)Het. Further preferred still are compounds in which R⁴ represents iodo, lower alkyl, lower alkynyl (which latter two groups are substituted and/or terminated by C(O)OR⁹ (wherein R⁹ represents H or C₁₋₆ alkyl)), N(H)Y(O)R¹⁷, N[Y(O)R¹⁷]₂, optionally substituted Het or NR¹²R¹³ (wherein R¹² and R¹³ together represent C₃-₅ alkylene interrupted by O or N—S(O)₂-(optionally substituted aryl)).

The present invention also encompasses the use of mimetics or bioisosteres of the above presented compounds.

Suitable PDE5 inhibitors for the use according to general formula (I) include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application W095/19978; the compounds disclosed in published international application WO 99/24433 and the compounds disclosed in published international application WO 93/07124.

The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Preferred pyrazolopyrimidinone PDE5 inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methlpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazlol[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazlol[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

Particularly preferred pyrazolopyrimidinones for use herein are: sildenafil (5-[2-ethoxy-5-(4-methyl-1-piperazinylsulphonyl)phenyl]-1-methyl-3-n-propyl-1,6-dihydro-7H-pyrazolo[4,3-d]pyrimidin-7-one), or 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one (1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine) or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one.

Especially preferred herein is sildenafil or pharmaceutically acceptable salts thereof.

It is to be understood that the PDE5 inhibitor may be a mimetic and/or chemical derivative of the above presented compounds. For example, in the case of sildenafil, this material may be in the form of sildenafil per se, a mimetic or a chemical derivative thereof.

### SILDENAFIL

Sildenafil is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one and is also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine.

Whilst any of the pharmaceutically acceptable salts of sildenafil may be used in accordance with the present invention, typically sildenafil is used as a citrate salt or as a mesylate salt, preferably the citrate salt.

According to a further aspect the present invention additionally provides for the use of the following further compounds for the treatment of PCOS:

(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; and/or

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433.

Still other PDE5 inhibitors include: the compound of example 11 of published international application WO93/07124 (EISAI); and compounds 3 and 14 from Rotella D P, J. Med. Chem., 2000, 43, 1257; 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidinecarboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

### SUBSTITUTED

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

### CHEMICAL MODIFICATION

In one embodiment of the present invention, the PDE5 inhibitor may be a chemically modified agent.

The chemical modification of a PDE5 inhibitor may either enhance or reduce hydrogen bonding interaction, charge interaction, hydrophobic interaction, Van Der Waals interaction or dipole interaction between the PDE5 inhibitor and the PDE5 enzyme.

In one aspect, an identified PDE5 inhibitor may act as a model (for example, a template) for the development of other compounds.

### PHARMACEUTICALLY ACCEPTABLE SALT

The PDE5 inhibitor may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc, diolamine, olamine, ethylenediamine, tromethamine, chloine, megulamine and diethanolamine salts. For reviews on suitable pharmaceutical salts see Berge et al J. Pharm. Sci., 66, 1-19 (1977); Gould P. L., International J. of Pharmaceutics, 33 (1986), 201-217; and Bighley et al, Encyclopedia of Pharmaceutical Technology, Marcel Dekker Inc., New York (1996), Vol. 13, page 453-497. A preferred salt is the sodium salt.

The pharmaceutically acceptable solvates of the pyrazolopyrimidinone PDE5 inhibitors of the invention include the hydrates thereof.

Herein, pyrazolopyrimidinone PDE5 inhibitors, their pharmaceutically acceptable salts, solvates and polymorphs, defined in any aspect of the invention (except intermediate compounds in chemical processes) are sometimes referred to as “compounds of the invention” or to as “agents of the invention”.

### POLYMORPHIC FORM(S)/ASYMMETRIC CARBON(S)

The PDE5 inhibitor may exist in polymorphic form.

The PDE5 inhibitor may contain one or more asymmetric carbon atoms and therefore exists in two or more stereoisomeric forms. Where a PDE5 inhibitor contains an alkenyl or alkenylene group, cis (E) and trans (Z) isomerism may also occur. The present invention includes the individual stereoisomers of the PDE5 inhibitors and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the PDE5 inhibitor or a suitable salt or derivative thereof. An individual enantiomer of the PDE5 inhibitor may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

### ISOTOPIC VARIATIONS

The present invention also includes all suitable isotopic variations of the PDE5 inhibitor or a pharmaceutically acceptable salt thereof. An isotopic variation of a PDE5 inhibitor of the present invention or a pharmaceutically acceptable salt thereof is defined as one in which at least one atom is replaced by an atom having the same atomic number but an atomic mass different from the atomic mass usually found in nature. Examples of isotopes that can be incorporated into the PDE5 inhibitor and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl respectively. Certain isotopic variations of the PDE5 inhibitor and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the PDE5 inhibitor and pharmaceutically acceptable salts thereof can generally be prepared by conventional procedures using appropriate isotopic variations of suitable reagents.

### PRODRUGS

It will be appreciated by those skilled in the art that the PDE5 inhibitor may be derived from a prodrug. Examples of prodrugs include entities that have certain protected group(s) and which may not possess pharmacological activity as such, but may, in certain instances, be administered (such as orally or parenterally) and thereafter metabolised in the body to form the PDE5 inhibitors which are pharmacologically active.

All protected derivatives and prodrugs of the PDE5 inhibitors of the present invention are included within the scope of the invention.

### PRO-MOIETIES

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the PDE5 inhibitors. Such prodrugs are also included within the scope of the invention.

### ANTAGONIST

The term inhibitor as used herein, for example with regard to PDE5i compounds and other additional active agents, in some instances may be regarded as being interchangeable with the term antagonist.

As used herein, the term “antagonist” means any agent that reduces the action of another agent or target. The antagonistic action may result from a combination of the substance being antagonised (chemical antagonism) or the production of an opposite effect through a different target (functional antagonism or physiological antagonism) or as a consequence of competition for the binding site of an intermediate that links target activation to the effect observed (indirect antagonism).

### AGONIST

As used herein the term “agonist” means any agent that enhances the action of or activates another agent or target. The term agonist includes a ligand that binds to receptors and thereby alters, typically increases, the proportion of them that are in an active form, resulting in a biological response.

### COMBINATIONS

As stated above, the present invention further comprises the combination of the PDE5 inhibitor for the treatment of PCOS with one or more additional active agents (for simultaneous, separate or sequential administration).

Thus, references herein to the use of PDE5 inhibitors for use according to the present invention also includes combination of PDE5 inhibitors with other additional (active) agents.

Such additional agent may be another PCOS drug as detailed hereinbefore, such as for example clomid.

Such additional agent may be another PDEi.

Combinations of PDE5 inhibitors, useful for the treatment of PCOS according to the present invention, with an additional agent are discussed in more detail below.

The method of the present invention may also be used in conjunction with hormone therapy. By way of example, the present invention may be used in conjunction with one or more hormones or steroids - such as those mentioned in WO-A-99/21562.

### ADDITIONAL ACTIVE AGENTS

Additional active agents suitable for use in the present invention include the following:

1) one or more naturally occurring or synthetic prostaglandins or esters thereof. Suitable prostaglandins for use herein include compounds such as alprostadil, prostaglandin E₁, prostaglandin E₀, 13, 14—dihydroprosta glandin E₁, prostaglandin E₂, eprostinol, natural synthetic and semisynthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁ α, 19-hydroxy PGA₁, 19-hydroxy —PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate; and/or

2) one or more α—adrenergic receptor antagonist compounds α-blockers. Suitable compounds for use herein include: the α-adrenergic receptor blockers as described in PCT application W099/30697 published on Jun. 14. 1998, the disclosures of which relating to a-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine (α₂-blocker), rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cardiotonic agent such as pirxamine; and/or

3) one or more NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso- N-acetyl penicilliamine (SNAP) S-nitroso-N-glutathione (SNO-GLU), N-hydroxy—L-arginine, amylnitrate, linsidomine, linsidomine chlorohydrate, (SIN-1) S-nitroso—N-cysteine, diazenium diolates,(NONOates), 1,5-pentanedinitrate, L-arginene, ginseng, zizphi fructus, molsidomine, Re—2047, nitrosylated maxisylyte derivatives such as NMI-678-11 and NMI-937 as described in published PCT application WO 0012075; and/or

4) one or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂; and/or

5) one or more dopaminergic agents, preferably apomorphine or a selective D2, D3 or D2/D₃agonist such as, pramipexole and ropirinol (as claimed in WO-0023056), L-Dopa or carbidopa, PNU95666 (as claimed in WO-0040226); and/or

6) one or more vasodilator agents. Suitable vasodilator agents for use herein include nimodepine, pinacidil, cyclandelate, isoxsuprine, chloroprumazine, halo peridol, Rec 15/2739, trazodone, and/or

7) one or more thromboxane A2 agonists; and/or

8) one or more ergot alkoloids; Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride; and/or

9) one or more compounds which modulate the action of natruretic factors in particular atrial naturetic factor (also known as atrial naturetic peptide), B type and C type naturetic factors such as inhibitors of neutral endopeptidase; and/or

10) one or more angiotensin receptor antagonists such as losartan; and/or

11) one or more substrates for NO-synthase, such as L-arginine; and/or

12) one or more calcium channel blockers such as amlodipine; and/or

13) one or more antagonists of endothelin receptors and inhibitors or endothelin-converting enzyme; and/or

14) one or more cholesterol lowering agents such as statins (e.g. atorvastatin/ Lipitor-trade mark) and fibrates; and/or

15) one or more antiplatelet and antithrombotic agents, e.g. tPA, uPA, warfarin, hirudin and other thrombin inhibitors, heparin, thromboplastin activating factor inhibitors; and/or

16) one or more insulin sensitising agents such as Rezulin, Avandia or Actos and hypoglycaemic agents such as, but not limited to, glipizide (sulfonylureas), metformin, or acarbose; and/or

17) one or more acetylcholinesterase inhibitors such as donezipil; and/or

18) one or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

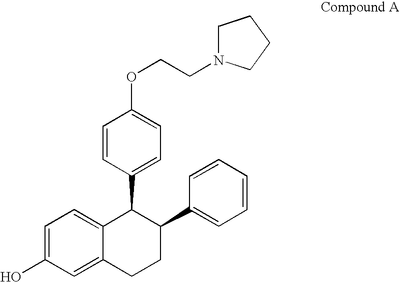

19)one or more of a further PDE inhibitor, more particularly a PDE 2, 4, 7 or 8 inhibitor, preferably PDE2 inhibitor, said inhibitors preferably having an IC50 against the respective enzyme of less than 100nM: and/or

20) one or more of an NPY (neuropeptide Y) inhibitor, more particularly NPY1 or NPY5 inhibitor, preferably NPYI inhibitor, preferably said NPY inhibitors (including NPY Y1 and NPY Y5) having an IC50 of less than 100 nM more preferably less than 50 nM, suitable NPY and in particular NPY1 inhibitor compounds are described in EP-A-1097718; and/or

21) one or more of vasoactive intestinal peptide (VIP), VIP mimetic, more particularly mediated by one or more of the VIP receptor subtypes VPAC1,VPAC or PACAP (pituitary adenylate cyclase activating peptide), one or more of a VIP receptor agonist or a VIP analogue (eg Ro-125-1553) or a VIP fragment, one or more of a o-adrenoceptor antagonist with VIP combination (eg lnvicorp, Aviptadil); and/or

22) one or more of a melanocortin receptor agonist or modulator or melanocortin ehancer, such as melanotan II, PT-14, PT-141 or compounds claimed in WO-09964002, WO-00074679, WO-09955679, WO-00105401, WO-00058361, WO-00114879, WO-00113112, WO-09954358 and/or

23) one or more of a serotonin receptor agonist, antagonist or modulator, more particularly agonists, antagonists or modulators for 5HTIA (including VML 670), 5HT2A, 5HT2C, 5HT3 and/or 5HT6 receptors, including those described in WO-09902159, WO-00002550 and/or WO-00028993; and/or

24) one or more of a testosterone replacement agent (inc dehydroandrostendione), testosternone (Tostrelle), dihydrotestosterone or a testosterone implant; and/or

25) one or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone); and /or

26) one or more of a modulator of transporters for noradrenaline, dopamine and/or serotonin, such as bupropion, GW-320659; and/or

27) one or more of a purinergic receptor agonist and/or modulator; and/or

28) one or more of a neurokinin (NK) receptor antagonist, including those described in WO-09964008; and/or

29) one or more of an opioid receptor agonist, antagonist or modulator, preferably agonists for the ORL-1 receptor; and/or

30) one or more of an agonist or modulator for oxytocin/vasopressin receptors, preferably a selective oxytocin agonist or modulator; and/or

31) one or more modulators of cannabinoid receptors; and/or

32) one or more of an NEP inhibitor, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; and/or

33) one or more compounds which inhibit angiotensin-converting enzyme such as enalapril, and one or more combined inhibitors of angiotensin-converting enzyme and neutral endopeptidase such as omapatrilat; and/or

34) one or more substrates for NO-synthase, i.e. L-arginine and/or; one or more calcium-channel blockers such as amlodipine; and/or

35) one or more antagonists of endothelin receptors and inhibitors of endothelin-converting enzyme; and/or

36) one or more cholesterol lowering agents e.g. statins and fibrates; antiplatelet and antithrombotic agents, e.g. tPA, uPA, warfarin, hirudin and other thrombin inhibitors, heparin, thromboplastin activating factor inhibitors; and/or

37) one or more L-DOPA and carbidopa and/or; one or more COX2 inhibitors and/or; pregabalene; and/or

38) gabapentene; and/or

39) one or more tricyclic antidepressants, e.g. amitriptiline; and/or

40) one or more non-steroidal anti-inflammatory agents and/or; one or more angiotensin-converting enzyme (ACE) inhibitors, e.g. quinapril; and/or

41) one or more anti-depressants (such as clomipramine and SSRIs (such as paroxetine and sertaline); and/or

42) one or more CNS active agents; and/or

43) one or more protein kinase C-p inhibitors such as LY333531; and/or 44) one or more activators of AMP-activated protein kinase such as 5-amino-4-imidazolecarboxamide ribonucleoside; and/or

45) insulin; and/or

46) weight loss agents such as sibutramine or orlistat; and/or

47) one or more dipeptidyl peptidase IV inhibitors such as NVP DPP728 or P32198; and/or

48) one or more glucagon antagonists such as NNC₂₅-2504; and/or

49) one or more agents that inhibit PTP1 B such as PTP1 12; and/or

50) one or more agents that reduce PTP1 B levels using antisense technology; and/or

51) one or more glycogen synthase kinase-3 inhibitors such as Chir98014; and/or

52) one or more GLP-1 agonists such as GLP1, NN-2211 or exendin 4; and/or

53) one or more PPAR-gamma agonists such as Rezulin, Avandia, Actos or CS011; and/or

54) one or more PPAR-alpha agonists such as fenofibrate; and/or 55) one or more dual PPAR-alpha/PPAR-gamma agonists such as farglitazar, rosiglitasone, pioglitazone, GW1929, DRF2725, AZ242 or KRP 297; and/or

56) one or more sorbitol dehydrogenase inhibitors such as CP-470711; and/or

57) one or more aldose reductase inhibitors such as zopolrestat, zenarestat, or fidarestat; and/or

58) one or more preparations of growth hormone or growth hormone secretagogues.

### KITS

The present invention also includes the use of kits that are useful in the method.

Typically the kit will comprise a pyrazolpyrimidinone PDE5 inhibitor, preferably sildenafil or a pharmaceutically acceptable salt thereof, in an effective amount and one or more of:

a. means for testing for PCOS

b. one or more pharmaceutically acceptable carrier, excipient or diluent

c. one or more additional active agents.

Although sildenafil is exemplified and claimed herein it is to be understood that the present invention additionally relates to the use of potent and preferably selective cGMP PDE51's for the treatment of PCOS in combination with an additional agent as detailed hereinbefore.

### PHARMACEUTICAL COMPOSITIONS

Although the PDE5 inhibitors can be administered alone, they will generally be administered in admixture with a suitable pharmaceutical excipient, diluent or carrier selected with regard to the intended route of administration and standard pharmaceutical practice.

Thus the present invention also provides a pharmaceutical composition comprising a therapeutically effective amount of the PDE5 inhibitor of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical compositions of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the PDE5 inhibitor is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the PDE5 inhibitor of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the agent drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the PDE5 inhibitors of the present invention are delivered systemically (such as orally, buccally, sublingually), more preferably orally.

Hence, preferably the PDE5 inhibitor is in a form that is suitable for oral delivery.

### ADMINISTRATION

The term “administered” includes delivery by viral or non-viral techniques. Viral delivery mechanisms include but are not limited to adenoviral vectors, adenoassociated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The PDE5 inhibitors of the present invention may be administered alone but will generally be administered as a pharmaceutical composition - e.g. when the PDE5 inhibitor is in admixture with a suitable pharmaceutical excipient, diluent or carrier selected with regard to the intended route of administration and standard pharmaceutical practice.

For example, the PDE5 inhibitor can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the PDE5 inhibitor may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, penile, vaginal, epidural, sublingual.

It is to be understood that not all of the active agents (i.e. the PDE5 inhibitor(s) and any additional agent(s)) need be administered by the same route. That is, some or all of the PDE5 inhibitor(s) and any additional agent(s) may be administered by different routes.

If the PDE5 inhibitor of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the PDE5 inhibitor; and/or by using infusion techniques.

For parenteral administration, the PDE5 inhibitor is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the PDE5 inhibitor of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the PDE5 inhibitor, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the PDE5 inhibitor and a suitable powder base such as lactose or starch.

Alternatively, the PDE5 inhibitors of the present invention can be administered in the form of a suppository or pessary, or it may be applied topically in the form of a gel, hydrogel, lotion, solution, cream, ointment or dusting powder. The PDE5 inhibitors of the present invention may also be dermally or transdermally administered, for example, by the use of a skin patch. They may also be administered by the pulmonary or rectal routes.

For application topically to the skin, the PDE5 inhibitor of the present invention can be formulated as a suitable ointment containing the PDE5 inhibitor suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the PDE5 inhibitor is administered orally which typically avoids systemic side effects.

Generally, in humans, oral administration of the PDE5 inhibitor is the preferred route, being the most convenient. In circumstances where the recipient suffers from a swallowing disorder or from impairment of drug absorption after oral administration, the drug may be administered parenterally, sublingually or buccally.

In one embodiment of the present invention there is provided a pharmaceutical medicament for use in the treatment of PCOS which is adapted for administration by mouth, said medicament comprising a PDE5 inhibitor having an IC50 less than 100 nanomolar and a selectivity over PDE3 of greater than 100.

Although reference has been made in this “Administration” section to the administration of the PDE5 inhibitor, it is to be understood that the administration techniques also apply to any additional agent administered.

### DOSE LEVELS

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The PDE5 inhibitor and/or the pharmaceutical composition of the present invention may be administered in accordance with a regimen of from 1 to 10 times per day, such as once or twice per day.

For oral and parenteral administration to humans, the daily dosage level of the PDE5 inhibitor may be in single or divided doses.

Depending upon the need, the PDE5 inhibitor may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Typically the daily oral dose may be, for instance, between 3-1500 mg, e.g. between 20-1000 mg, and preferably 50-300 mg.

The dosage of PDE5 inhibitor for oral, buccal, sublingual or parenteral administration may, for example, be in the range of from 1 to 500 mg for administration up to three times a day. For oral and parenteral administration to human patients, the daily dosage level of the PDE5 inhibitor will usually be from 5 to 500 mg (in single or divided doses). In the case of sildenafil, a preferred dose is in the range 5 to 100 mg (e.g. 5, 10, 20, 40 and 80 mg) which can be administered once, twice or three times a day (preferably once). However, as stated above, the precise dose will be as determined by the prescribing physician and will depend on various factors such as the age and weight of the patient and severity of the symptoms.

Thus, for example, tablets or capsules of the PDE5 inhibitor may contain from 5 to 250 mg (e.g. 10 to 100 mg) of the PDE5 inhibitor for administration singly or two or more at a time, as appropriate. The physician in any event will determine the actual dosage which will be most suitable for any individual patient and it will vary depend on factors such as the age, weight and response of the particular patient. The above dosages are exemplary of the average case. There can, of course, be individual instances where higher or lower dosage ranges are merited and such are within the scope of this invention.

Aerosol or dry powder formulations are preferably arranged so that each metered dose or “puff” contains from 1 to 50 mg of the PDE5 inhibitor, for delivery to the patient. The overall daily dose with an aerosol will generally be in the range of from 1 to 50 mg which may be administered in a single dose or, more usually, in divided doses throughout the day.

Suitable doses of the PDE5 inhibitor will include those which allow a satisfactory therapeutic ratio between the treatment of PCOS, and the induction of emesis or other side effects.

### FORMULATION

The PDE5 inhibitors of the present invention may be formulated into a pharmaceutical composition, such as by mixing with one or more of a suitable carrier, diluent or excipient, by using techniques that are known in the art.

The following present some non-limiting examples of formulations.

Formulation 1: A tablet is prepared using the following ingredients:

| | weight/mg |
|---|---|
| Sildenafil citrate | 250 |
| Cellulose, microcrystalline | 400 |
| Silicon dioxide, fumed | 10 |
| Stearic acid | 5 |
| Total | 665 |

the components are blended and compressed to form tablets each weighing 665 mg.

Formulation 2: An intravenous formulation may be prepared as follows:

| | |
|---|---|
| Sildenafil citrate | 100 mg |
| Isotonic saline | 1,000 ml |

Formulation 3: A tablet is prepared using the following ingredients:

Sildenafil citrate (50 mg) is blended with cellulose (microcrystalline), silicon dioxide, stearic acid (fumed) and the mixture is compressed to form tablets.

### BIOAVAILABILITY

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 μg/ml. Solubility can be determined by standard procedures known in the art such as described in Adv. Drug Deliv. Rev. 23, 3-25,1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of —2 to +4, more preferably −1 to +2. The log D can be determined by standard procedures known in the art such as described in J. Pharm. Pharmacol. 1990,42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶cms⁻¹, more preferably greater than 5×10⁻⁶cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stability in the assay system that is commensurate with a hepatic extraction of less then 0.5. Examples of assay systems and data manipulation are described in Curr. Opin. Drug Disc. Devel., 201, 4, 36-44, Drug Met. Disp.,2000, 28,1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226.

### PDE5 INHIBITOR ASSAYS

PDE action potency values referred to herein may be determined by the following assays:

Phosphodiesterase (PDE) Inhibitory Activity

Preferred PDE compounds suitable for use in accordance with the present invention are potent and selective PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham pic under product code TRKQ7090/7100. In summary, the effect of PDE inhibitors was investigated by assaying a fixed amount of enzyme in the presence of varying inhibitor concentrations and low substrate, (cGMP or cAMP) in a 3:1 ratio unlabelled to [³H]-labeled at a conc ˜1/3 Km) such that IC_{50 ≅K}ᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC50 values obtained using the ‘Fit Curve’ Microsoft Excel extension.

Functional Activity

This can be assessed in vitro by determining the capacity of a PDE5 inhibitor of the invention to enhance sodium nitroprusside or electric field stimulationinduced relaxation of pre-contracted rabbit corpus cavernosum tissue strips, as described by S. A. Ballard et al. (Brit. J. Pharmacol., 1996, 118 (suppl.), abstract 153P) or S. A. Ballard et al. (J. Urology, 1998, vol.159, 2164-2171).

### IN VITRO PDE INHIBITORY ACTIVITIES

In vitro PDE inhibitory activities against cyclic guanosine 3′,5′-monophosphate (cGMP) phosphodiesterases can be determined by measurement of their IC50 values (the concentration of compound required for 50% inhibition of enzyme activity).

The required PDE enzymes can be isolated from a variety of sources, including human corpus cavernosum, human and rabbit platelets, human cardiac ventricle, human skeletal muscle and human and canine retina, essentially by the method of W. J. Thompson and M. M. Appleman (Biochem., 1971, 10, 311).

Likewise, other enzymes can be isolated from a variety of sources. These other enzymes can then be used to determine the selectivity of the PDE5 inhibitor for use in the present invention.

By way of example, a cGMP-specific PDE (PDE5) and a cGMP-inhibited cAMP PDE (PDE3) can be obtained from human corpus cavernosum or human platelets; a cGMP-stimulated PDE (PDE2) can be obtained from human corpus cavernosum and human platelets; a calcium/calmodulin (Ca/CAM)-dependent PDE (PDE1) can be obtained from human cardiac ventricle; a cAMP-specific PDE (PDE4) can be obtained from human skeletal muscle and human recombinant, expressed in SF9 cells; and a photoreceptor PDE (PDE6) can be obtained from human or canine retina. Phosphodiesterases 7-11 can be generated from full length human recombinant clones transfected into SF9 cells.

### EXAMPLES

The invention will now be further described only by way of example.

### Functional Activity of Pyrazolopyrimidinones

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

### In vivo Activity of Pyrazolopyrimidinones

Pyrazolopyrimidinone compounds as described herein, and sildenafil in particular were screened in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

### Results

The following experiments, 1 and 2, demonstrate the activity of pyrazolopyrimidinone cyclic guanosine 3′,5′-monophosphate phosphodiesterase type five (cGMP PDE V) inhibitors in the treatment of improved uterine and ovarian blood flow as well as increasing progesterone levels which as detailed hereinbefore are all clinical parameters associated with polycystic ovary syndrome.

### Experiment 1

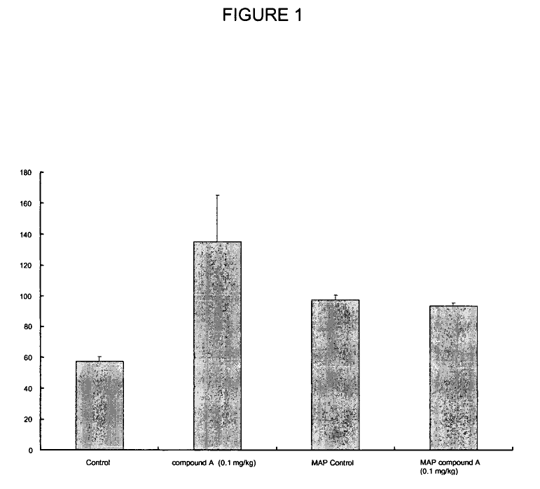

A model of uterine blood flow was established to assess the effects of pyrazolopyrimidinone PDE V inhibition on uterine flow and mean arterial blood pressure (MAP). Mini-pigs weighing approximately 30 kg were modified surgically. As a means of quantifying real time uterine blood flow a Transoinc blood flow probe was placed around either the left or right uterine artery. Catheters were inserted into a external jugular vein and a carotid artery for blood sampling/administration of compound and measurement of mean arterial blood pressure (MAP) respectively. Test compound was administered intravenously either during oestrous or the luteal phase of the reproductive cycle and the effects of PDE V inhibition on uterine blood flow and MAP were measured. The results are shown in FIG. 1. The Y-axis for the two left hand bars is uterine blood flow (ml/min.), and for the two right hand bars is MAP (mmHg).

[image]

[image]

[image]

[image]

[image]

[image]

Oestrous was synchronised using two intramuscular injections of PGF2a (Lutalyse™) administered 11 days apart.

The pyrazolopyrimidinone PDE V inhibitor was administered intra-vaginally twice daily from day −3 of oestrus until day 6 of the following oestrus (30 days in total). Blood samples were collected daily, plasma was prepared as soon as possible and stored at −20° C. and then analysed for progesterone.

At the completion of the study (ie day 6 of the second oestrous period) the cattle were euthanised and the reproductive tract collected. A cross section of the left and right horns of each uterus was collected and stored in 10% Formalin and ten analysed histologically.

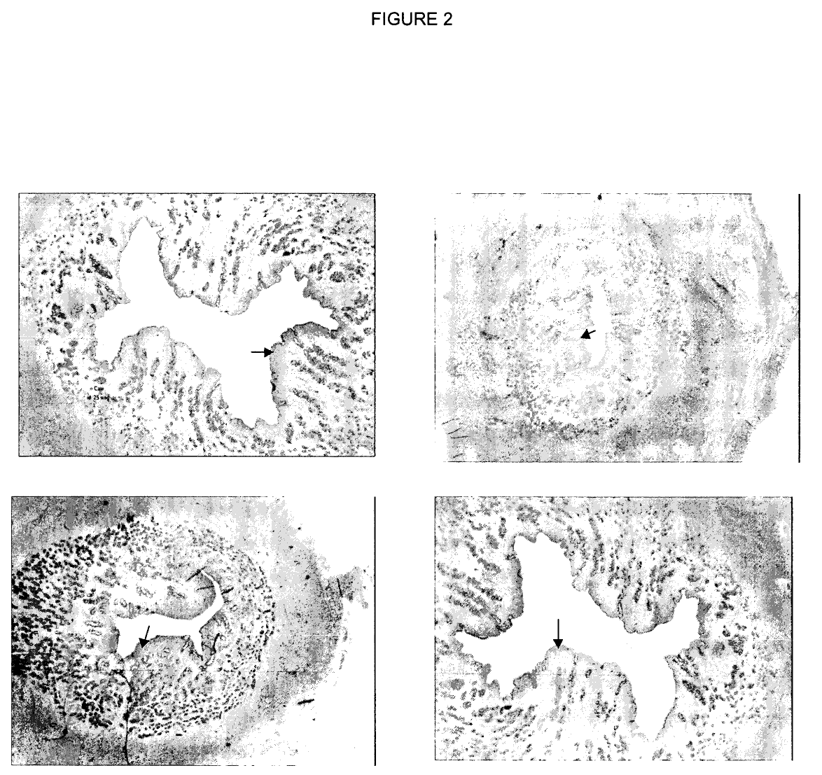

FIG. 2

[image]

[image]

[image]

[image]

[image]

[image]

FIG. 2. Cross-section of the unterine horn of control animals. Arrows depict endometrial epithelial layer.

[image]

[image]

[image]

[image]

[image]

[image]

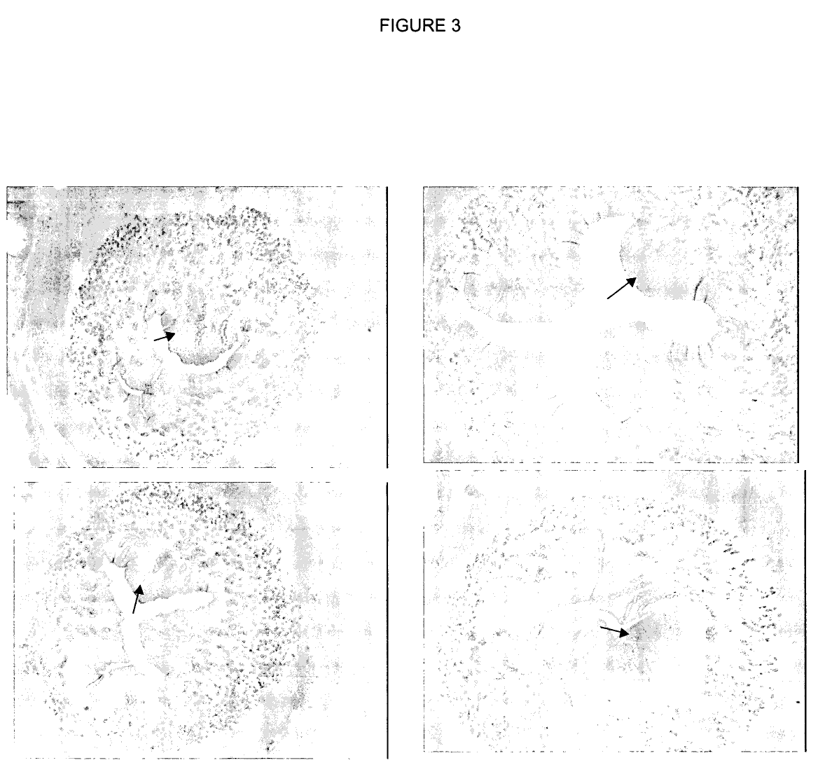

FIG. 3 Cross-section of the uterine horn of animals treated with a pyrazolopyrimidinone PDE V inhibitor (compound A). Arrows depict endometrial epithelial.

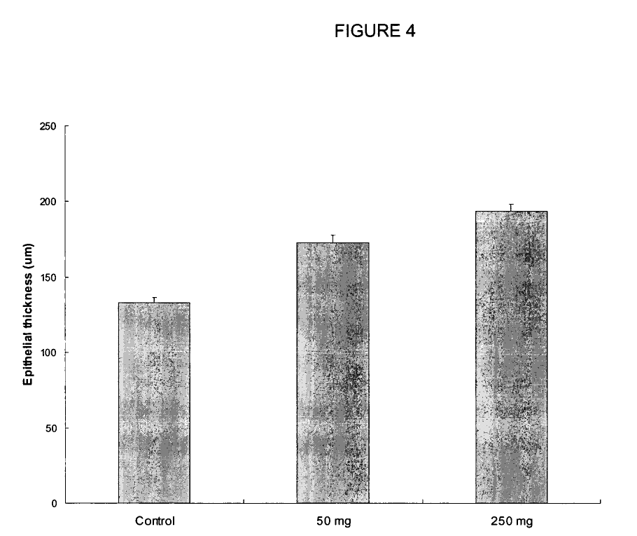

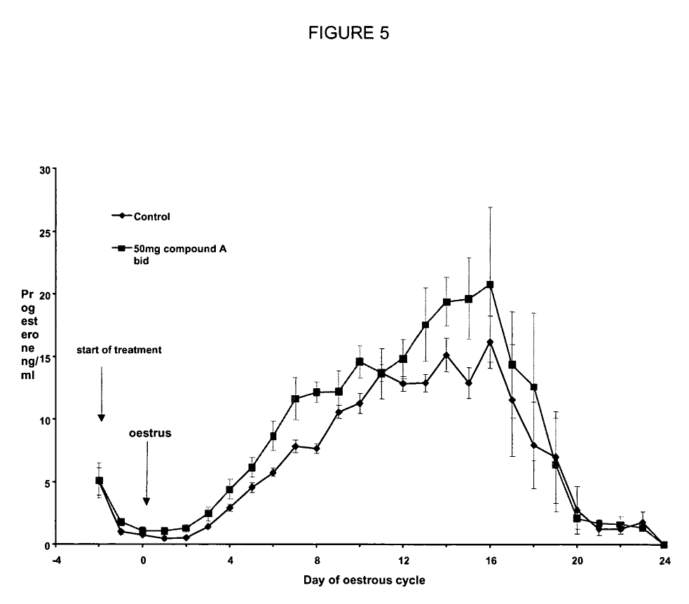

FIG. 4

[image]

[image]

[image]

[image]

[image]

[image]

FIG. 4. Pyrazolpyrimidinone PDE V inhibitor (compoun A) increases endometrial epithelial thickness relative to control animals.

[image]

[image]

[image]

[image]

[image]

[image]

It is concluded from the results of experiments 1 and 2 described above that PDE V inhibition via treatment with a pyrazolpyrimidinone PDE V inhibitor promotes improvements in uterine blood flow and progesterone levels which are key clinical parameters associated with PCOS in mammals.

The results of experiment 1 demonstrate that treatment with a pyrazolopyrimidinone PDE V inhibitor leads to increased levels of uterine blood flow. It is proposed herein that such PDE V inhibition of uterine blood is associated with improved fertility in subjects with PCOS.

In particular the results of experiment 2 show that treatment with a pyrazolopyrimidinone PDE V inhibitor leads to increased levels of progesterone. It is proposed herein that such PDE V inhibition promotes ovarian blood flow which in turn results in enhanced nutrient supply to the to the ovary and increased progesterone levels. Thus according to a further aspect the present invention additionally provides for the use of pyrazolopyrimidinone PDE V inhibitors for the treatment of conditions where a low progesterone level is implicated. Such conditions are commonly referred to as low progesterone disorders.

Low as defined herein means a female having progesterone level(s) during the luteal phase of the menstrual cycle which is inferior to the normal luteal level(s) expected in a pre-menopausal female mammal of her age. Examples of low progesterone disorders potentially treatable according to this aspect of the invention include poor endometrial gland function, short luteal phases, short menstrual cycles, pre-menstrual syndromes and recurrent abortion. Suitable cGMP PDE5i's for such treatment are those described hereinbefore and particularly include potent and selective cGMP PDE5i's. Especially preferred for such treatment is sildenafil. Whilst any the chosen PDE5i and sildenafil in particular can be formulated and dosed for the treatment of low progesterone disorders according to any of the means described herein before, oral and intra-vaginal dosing are preferred, intra-vaginal being particularly preferred.

Maturation of the graphian follicle leading to ovulation is the key missing event in infertility due to PCOS. It is further proposed herein that enhanced blood supply to the ovary leads to improved delivery of important hormonal signals such follicular stimulating hormone (FSH) and lutenizing hormone (LH) along with nutrients supply responsible for ovulation. The result of such improved delivery of key hormonal signals is an enhanced maturation of a dominant follicle leading to ovulation. In addition, enhanced blood flow prior to or following ovulation would enhance the formation of corpus leutum (formed from the remnants of ovulated follicle) which is responsible for the production of progesterone. The premature death of corpus leutum may decrease chances of implantation, and hence the enhance blood flow could extend the life of corpus leutum, increase progesterone production, and increase the chances of fertility.

Thus according to a further aspect the present invention provides the use of PDE V inhibitors, particularly pyrazolopyrimidinone PDE V inhibitors, and especially sildenafil or a pharmaceutically acceptable salt thereof for enhanced or improved ovulation.

Thus according to a yet further aspect the present invention provides the use of a PDE V inhibitors, particularly pyrazolopyrimidinone PDE V inhibitor, and especially sildenafil or a pharmaceutically acceptable salt thereof for improved follicular maturation

Thus according to a yet further aspect the present invention provides the use of a PDE V inhibitors, particularly pyrazolopyrimidinone PDE V inhibitor, and especially sildenafil or a pharmaceutically acceptable salt thereof for the improved formation of corpus leutum and subsequently increase progesterone concentrations.

It is also proposed herein that as subjects with PCOS have insulin resistance and are infertile consequently treatment with PDE V inhibitors and pyrazolopyrimidinone PDE V inhibitors in particular, and especially sildenafil and pharmaceutically acceptable salts thereof may have at least additive effects and potentially synergistic benefits for subjects with PCOS by virtue of addressing their glucose metabolism and increasing uterine blood flow which in turn leads to glucose homeostasis and fertility.

Experiments 3, 4 and 5 demonstrate the effect of specific selective pyrazolopyrimidinone PDE V inhibitors, and sildenafil in particular on factors affecting PCOS in animals—Effects on Plasma Glucose and Serum Triglyceride Levels in ob/ob Mice.

Biological Data

Experimental Protocol

Test Compounds:

The selective pyrazolopyrimidinone PDEV inhibitor compounds to be tested were solubilized in 10% DMSO/0.1% pluronics and dosed via oral gavage using mouse oral feeding needles (20 gauge, Popper & Sons, Inc., New Hyde Park, N.Y.). A volume of 4 ml/kg weight was administered for each dose. Compounds were tested at doses ranging from 1-50 mg/kg. Alternatively, the test selective pyrazopyrimidinone PDEV inhibitor compound was administered in the drinking water and found to produce similar reductions in plasma glucose and triglycerides to the reductions observed for the same compound when administered by oral gavage.

Experimental Animals:

Male oblob mice obtained from Jackson Laboratories (Bar Harbor, Me.) were used in the studies at 6 to 10 weeks of age. Mice were housed five per cage and allowed free access to D11 mouse chow (Purina, Brentwood, Mo.) and water.

Experimental Protocol:

Mice were allowed to acclimate to the Pfizer animal facilities for 1 week prior to the start of the study. On day one, retro-orbital blood samples were obtained and plasma glucose was determined as described hereinafter. Mice were then sorted into groups of five such that mean plasma glucose concentrations for each group did not differ. On day one, mice were dosed with vehicle or a test selective pyrazolopyrimidinone PDEV inhibitor compound only in the afternoon. Subsequently, mice were dosed twice a day on day 2-4 in the morning and in the afternoon. On day 5, the mice received an a.m. dose and bled 3 hours later for plasma preparation for glucose and triglyceride analysis as described below. Alternatively, test selective pyrazolopyrimidinone PDEV inhibitor compound was administered in the drinking water commencing on the afternoon of day 1 and continuing through day 5, when mice were then bled for plasma preparation for glucose and triglyceride analysis as described below. Terminal plasma samples were collected on day 5 following the retro-orbital sinus bleed as described below. Body weight was measured on days 1 and 5 of the study, and food consumption was assessed over the 5 day period.

Terminal Bleed and Tissue Collection:

On the morning of the last day of the study mice were dosed with test pyrazolopyrimidinone PDEV compound or vehicle at approximately 8:00 am. Three hours after dosing, 25 μL of blood was obtained via the retro-orbital sinus and added to 100 μL of 0.025% heparinized-saline in Denville Scientific microtubes. The tubes were spun at the highest setting in a Beckman Microfuge 12 for 2 minutes. Plasma was collected for plasma glucose and triglyceride determination. The mice were then sacrificed by decapitation and ˜1 ml of blood was collected in Becton-Dickinson Microtainer brand plasma separator tubes with lithium heparin. The tubes were spun in a Beckman Microfuge 12 at the maximum setting for five minutes. Plasma was collected in 1.5 ml Eppendorf tubes and snap frozen in liquid nitrogen. Plasma samples were stored at −80° C. until analyzed.

Metabolite and Hormone Analysis:

Plasma glucose and triglycerides were measured using the Alcyon Clinical Chemistry Analyzer (Abbott Laboratories, Abbott Park, Ill.) using kits supplied by Abbott. Plasma cGMP was measured using the Biotrak enzymeimmunoassay system by Amersham (Piscataway, N.J.). Via a similar technique the plasma insulin can be assessed by the Mercodia ELISA Insulin kit by ALPCO (Uppsala, Sweden). All assays were conducted according to instructions provided by the manufacturers.

Statistical Analysis:

Comparisons between drug treatments and appropriate vehicles were done by Student's t-test.

Results (Summary):

Pyrazolopyrimidinone PDE V inhibitors and sildenafil in particular have been demonstrated to reduce the plasma glucose and serum triglyceride levels produced by ob/ob mice in accordance with the biological test methods detailed hereinbefore.

Experiment 3

Table 1 illustrates the changes in plasma glucose levels over a 5 day period observed with sildenafil and selective pyrazolopyrimidinone PDE V inhibitor B.

Selective PDE5 Compound A: 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (sildenafil)

Selective PDE5 Compound B: 5-(2-ethoxy-5-morpholinoacetylphenyl)-1-methyl-3-n-propyl-1,6-dihydro-7Hpyrazolo[4,3-d]pyrimidin-7-one

TCBLE 1

| | Changes in Plasma |
|---|---|
| | Glucose Concentrations |
| | (mg/dl) |
| Vehicle | −9 ± 22 |
| PDE5 A - 10 mg/kg | −115 ± 34* |
| PDE5 A - 50 mg/kg | −105 ± 25* |
| PDE5 B - 25 mg/kg | −97 ± 32* |

The data in Table 1 are presented as mean±standard error of the mean. These numbers reflect absolute decreases in plasma glucose levels. Significant differences from the Vehicle control are indicated as *p<0.05.

Experiment 4

Table 2 illustrates the change in plasma cGMP and plasma triglyceride levels in ob/ob mice observed with the test selective PDE5 inhibitor compounds A and B.

TCBLE 2

| | Plasma cGMP Level | Plasma Triglyceride |
|---|---|---|
| | (mg/dl) | Level (mg/dl) |
| Vehicle | 9.8 ± 0.5 | 178 ± 16 |
| PDE5 A - 10 mg/kg | 48.3 ± 19.0\hat{ } | 163 ± 10 |
| PDE5 B - 25 mg/kg | 30.7 ± 3.3** | 143 ± 7\hat{ } |

The data in Table 2 are presented as mean±standard error of the mean. Significant differences from the Vehicle control as indicated as \hat{ }p<0.1, *p<0.05, **p<0.01.

Experiment 5

Table 3 illustrates the reduction in plasma glucose levels over a 5 day period observed with selective a PDE5 inhibitor compound administered in the drinking water of the mice.

Selective PDE5 Compound C: sildenafil

| | TCBLE 3 |
|---|---|
| | Changes in Plasma Glucose |
| | Concentrations (mg/dl) |
| Vehicle | 25 ± 25 |
| PDE5 C - 9 mg/kg | −27 ± 34 |
| PDE5 C - 22 mg/kg | −15 ± 27 |
| PDE5 C - 45 mg/kg | −36 ± 22\hat{ } |

The data in Table 3 are presented as mean±standard error of the mean. Positive values in this table reflect a decrease in plasma glucose level. Significant differences from the Vehicle control are indicated as \hat{ }p<0.1.

Experiment 6

Table 4 illustrates the triglyceride levels in ob/ob mice treated with the test selective PDE5 inhibitor compound C administered in the drinking water of the mice.

| | TCBLE 4 |
|---|---|
| | Plasma Triglyceride |
| | Level (mg/dl) |
| Vehicle | 204 ± 13 |
| PDE5 C - 9 mg/kg | 163 ± 14* |
| PDE5 C - 22 mg/kg | 212 ± 20 |
| PDE5 C - 45 mg/kg | 151 ± 10** |

The data in Table 4 are presented as mean±standard error of the mean. Significant differences from the Vehicle control as indicated as *p<0.05, **p<0.01.

Taken together, these experimental results in the hyperglycemic, insulinresistant ob/ob mouse suggest that selective PDE5 inhibition, in particular via treatment with sildenafil improves metabolic parameters associated with PCOS as detailed hereinbefore.

Further these results suggest that treatment with selective PDE5 inhibitors such as sildenafil can result in decreases in plasma glucose concentrations. As detailed herein before decreases in plasma glucose concentrations are consistent with an improvement in insulin resistance which is a clinical parameter of the PCOS, and, as further detailed hereinbefore such improvements, in subjects with PCOS would manifest as improvements in for example haemoglobin A1c.

These results also suggest that treatment with selective PDE5 inhibitors and sildenafil in particular can result in improvements in serum lipid levels. As detailed herein before an improvement in serum lipid levels (such as in triglyceride levels) is consistent with an improvement in insulin resistance which is a clinical parameter of PCOS. Such improvements, in subjects with PCOS(as defined herein) would manifest as improvements in for example dyslipidemia (hypertriglyceridaemia).

These results in the hyperglycemic, insulin-resistant ob/ob mouse additionally suggest that continuous treatment with a selective PDE5 inhibitor such as sildenafil can improve metabolic parameters associated with PCOS in 5 days or less.

The present invention additionally comprises the combination of a selective pyrazolopyrimidinone PDEV inhibitor, and sildenafil in particular and a glucoselowering agent for the treatment of PCOS. In particular said combined treatment is effected by the oral route.

According to a further aspect the present invention provides a combination therapy suitable for use in the treatment of the PCOS wherein said combination comprises a selective pyrazolopyrimidinone cGMP PDEV inhibitor, especially sildenafil with an additional agent active as defined hereinbefore and preferably one or more of: insulin lowering agents such as, Metformin, PPAR-gamma; bromocriptine; cimetidine; androgen biosynthesis inhibitors; 5-alpha reductase inhibitors such as finasteride; androgen receptor antagonists such as spironolactone, cyproterone acetate or flutamie; glucocorticoids; GnRH analogues in combination with oral contraceptives; clomid.

According to a yet further aspect said combination treatment(s) is/are effected orally and further may be in the form of a kit.

Taken together, the results from all the animal experiments detailed hereinbefore are consistent with improvements in clinical parameters associated with the PCOS. That is, improvements in triglycerides, as well as improvement in glucose levels, and improved uterine and ovarian blood flow and improved progesterone levels support the activity of potent and selective pyrazolopyrimidinone PDEV inhibitor compounds, and especially sildenafil and pharmaceutically acceptable salts thereof for treatment of PCOS.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry and biotechnology or related fields are intended to be within the scope of the following claims.

1. Use of sildenafil or a pharmaceutically acceptable salt, solvate or prodrug thereof in the preparation of a medicament for the treatment of polycystic ovary syndrome (PCOS).

2. A method of treating PCOS in an individual in need of treatment which comprises administering to said individual an effective amount of sildenafil or a pharmaceutically acceptable salt thereof.

3. A pharmaceutical composition for use in the treatment of PCOS comprising sildenafil or a pharmaceutically acceptable salt thereof admixed with a pharmaceutically acceptable carrier, diluent or excipient.

4. A pharmaceutical combination for simultaneous, separate or sequential administration, for the treatment of PCOS in an individual in need of treatment, comprising sildenafil or a pharmaceutically acceptable salt thereof and one or more additional active agents.

5. A method of preparing a pharmaceutical composition for use in the treatment of PCOS comprising admixing sildenafil or a pharmaceutically acceptable salt thereof with a pharmaceutically acceptable carrier, diluent or excipient.

6. Use of a pyrazopyrimidinone cGMP PDE5 inhibitor having general formula (I): 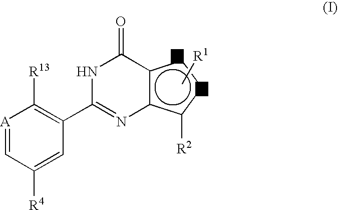
wherein:
A is CH or N;
R¹ is H, C₁ to C₆ alkyl, C₃ to C₆ alkenyl, C₃ to C₆ cycloalkyl, C₃ to C₆ cycloalkenyl, or C₁-C₃ perfluoroalkyl, wherein said alkyl group may be branched or straight chain and wherein said alkyl, alkenyl, cycloalkyl or perfluoroalkyl group is optionally substituted by; one or more substituents selected from: hydroxy; C₁ to C₄ alkoxy; C₃ to C₆ cycloalkyl; C₁-C₃ perfluoroalkyl; phenyl substituted with one or more substitutents selected from C₁ to C₃ alkyl, C₁ to C₄ alkoxy, C₁ to C₄ haloalkyl or C₁ to C₄ haloalkoxy wherein said haloalkyl and haloalkoxy groups contain one or more halo atoms, halo, CN, NO₂, NHR¹¹, NHSO₂R¹², SO₂R¹², SO₂NHR¹¹, COR¹¹, CO₂R¹¹ wherein R¹¹ is H, C₁ to C₄ alkyl, C₂ to C₄ alkenyl, C₁ to C₄ alkanoyl, C₁ to C₄ haloalkyl or C₁ to C₄ haloalkoxy and wherein R¹² is C₁ to C₄ alkyl, C₂ to C₄ alkenyl, C₁ to C₄ alkanoyl, C₁ to C₄ haloalkyl or C₁ to C₄ haloalkoxy; NR⁷R⁸, CONR⁷R⁸ or NR⁷COR¹¹ wherein R⁷ and R⁸ are each independently selected from H, C₁ to C₄ alkyl, C₂ to C₄ alkenyl, C₁ to C₄ alkoxy, CO₂R⁹, SO₂R⁹ wherein said alkyl, alkenyl or alkoxy groups are optionally substituted by NR⁵R⁶, C₁ to C₄ haloalkyl or C₁ to C₄ haloalkoxy and wherein R⁹ is H, hydroxy C₂ to C₃ alkyl, C₁ to C₄ alkanoyl or C₁ to C₄ alkyl which is optionally substituted with phenyl wherein said phenyl group is optionally substituted by one or more substituents selected from C₁ to C₄ alkyl optionally substituted by C₁ to C₄ haloalkyl or C₁ to C₄ haloalkoxy, C₁ to C₄ alkoxy, halo, CN, NO₂, NHR¹¹, NHSO₂R¹², SO₂R¹², SO₂NHR¹¹, COR¹¹ or CO₂R¹¹; Het¹; Het² or Het³; or R¹ is Het⁴ or phenyl wherein said phenyl group is optionally substituted by one or more substituents selected from C₁ to C₄ alkyl, C₂ to C₄ alkenyl, C₁ to C₄ alkoxy, halo, CN, CF₃, OCF₃, NO₂, NHR¹¹, NHSO₂R¹², SO₂R¹², SO₂NHR¹¹, COR¹¹, CO₂R¹¹;
R² is H, C₁ to C₆ alkyl, C₃ to C₆ alkenyl or (CH₂)ₙ(C₃ to C₆ cycloalkyl) wherein n is 0, 1 or 2 and wherein said alkyl or alkyenyl group is optionally substituted with one or more fluoro substituents;
R¹³ is OR³ or NR⁵R⁶;
R³ is C₁ to C₆ alkyl, C₃-C₆ alkenyl, C₃-C₆ alkynyl, C₃-C₇ cycloalkyl, C₁-C₆ perfluoroalkyl or (C₃-C₆ cycloalkyl)C₁-C₆ alkyl optionally substituted with one or two substituents selected from C₃ to C₅ cycloalkyl, hydroxy, C₁ to C₄ alkoxy, C₃-C₆ alkenyl, C₃-C₆ alkynyl, benzyloxy, NR⁵R⁶, phenyl, Het¹, Het², Het³ or Het⁴ wherein the C₁ to C₆ alkyl and C₁ to C₄ alkoxy groups may optionally be terminated by a haloalkyl group such as CF₃; C₃ to C₆ cycloalkyl; Het¹, Het², Het³ or Het⁴;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; hydroxy C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; or phenyl or heterocyclyl either of which is optionally substituted with methyl; or R⁴ is a pyrrolidinylsulphonyl, piperidinosulphonyl, morpholinosulphonyl, or piperazin-1-ylsulphonyl group having a substituent, R¹⁰ at the 4-position of the piperazinyl group wherein said piperazinyl group is optionally substituted with one or two C₁ to C₄ alkyl, C₁ to C₃ alkoxy, NR⁷R⁸ or CON R⁷R⁸ groups and is optionally in the form of its 4-N-oxide;
R⁵ and R⁶ are each independently selected from H and C₁ to C₄ alkyl optionally substituted with C₃ to C₅ cycloalkyl or C₁ to C₄ alkoxy, or, together with the nitrogen atom to which they are attached, form an azetidinyl, pyrrolidinyl, piperidinyl, morpholinyl, 4-(NR⁹)-piperazinyl or imidazolyl group wherein said group is optionally substituted with methyl or hydroxy;
R¹⁰ is H; C₁ to C₆ alkyl, (C₁-C₃ alkoxy) C₂-C₆ alkyl, hydroxy C₂-C₆ alkyl, (R⁷R⁸N)C₂-C₆ alkyl, (R⁷R⁸NCO)C₁-C₆ alkyl, CONR⁷R⁸, CSNR⁷R⁸ or C(NH)NR⁷R⁸ optionally substituted with one or two substituents selected from hydroxy, NR⁵R⁶, CONR⁵R⁶, phenyl optionally substituted with C₁ to C₄ alkyl or C₁ to C₄ alkoxy; C₂ to C₆ alkenyl or Het⁴;
Het¹ is an N-linked 4-, 5- or 6-membered nitrogen-containing heterocyclic group optionally containing one or more further heteroatoms selected from S, N or O;
Het² is a C-linked 5-membered heterocyclic group containing an O, S or N heteroatom optionally containing one or more heteroatoms selected from O or S;
Het³ is a C-linked 6-membered heterocyclic group containing an O or S heteroatom optionally containing one or more heteroatoms selected from O, S or N or Het³ is a C-linked 6-membered heterocyclic group containing three N heteroatoms;
Het⁴ is a C-linked 4-, 5- or 6-membered heterocyclic group containing one, two or three heteroatoms selected from S, O or N; and wherein any of said heterocyclic groups Het¹, Hef, Het³ or Het⁴ may be saturated, partially unsaturated or aromatic and wherein any of said heterocyclic groups may be optionally substituted with one or more substituents selected from C₁ to C₄ alkyl, C₂ to C₄ alkenyl, C₁ to C₄ alkoxy, halo, CO₂R¹¹, COR¹¹, SO₂R¹² or NHR¹¹ and/or wherein any of said heterocyclic groups is benzo-fused.
or wherein when R¹³ represents OR³ or R³NR⁵; R¹ represents Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R² represents H, halo, cyano, nitro, OR⁶, OC(O)R⁷, C(O)R⁸, C(O)OR⁹, C(O)NR¹⁰R¹¹, NR¹²R¹³, SO₂NR¹⁴R¹⁵, lower alkyl, Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R³ represents H, lower alkyl, alkylHet or alkylaryl, which latter three groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R⁴ represents H, halo, cyano, nitro, halo(loweralkyl), OR⁶, OC(O)R⁷, C(O)R⁸, C(O)OR⁹, C(O)NR¹⁰R¹¹, NR¹²R¹³, NR¹⁶Y(O)R¹⁷, SOR¹⁸, SO₂R¹⁹R²⁰, C(O)AZ, lower alkyl, lower alkenyl, lower alkynyl, Het, alkylHet, aryl, alkylaryl, which latter seven groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; Y represents C or S(O), wherein one of R¹⁶ and R¹⁷ is not present when Y is S(O); A represents lower alkylene; Z represents OR⁶, halo, Het or aryl, which latter two groups are both optionally substituted with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, N0R¹²R¹³ and SO₂NR¹⁴R¹⁵; R⁵, R⁶, R⁷, R⁸, R⁹, R¹⁸, R¹⁹ and R²⁰ independently represent H or lower alkyl; R¹⁰ and R¹¹ independently represent H or lower alkyl, which latter group is optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰OR¹¹, NR¹²R¹³ and SO₂NR¹⁴R¹⁵ or Het or aryl optionally substituted with one or more of said latter eleven groups or one of R¹⁰ and R¹¹ may be lower alkoxy, amino or Het, which latter two groups are both optionally substituted with lower alkyl; R¹² and R¹³ independently represent H or lower alkyl or one of R¹² or R¹³ may be C(O)-lower alkyl or C(O)Het in which Het is optionally substituted with lower alkyl; R¹⁴ and R¹⁵ independently represent H or lower alkyl or R¹⁴ and R¹⁵, together with the nitrogen atom to which they are bound, form a heterocyclic ring; R¹⁶ and R¹⁷ independently represent H or lower alkyl or one of R¹⁶ and R¹⁷ may be Het or aryl, which latter two groups are both optionally substituted with lower alkyl; Het represents an optionally substituted four to twelve membered heterocyclic group, which may be aromatic or non-aromatic, which may contain one or more double bonds, which may be mono- or bi-cyclic and which contains one or more heteroatoms selected from N, S and O;
or a pharmaceutically acceptable salt, solvate, mimetic or bioisostere of any thereof in the manufacture of a medicament for the treatment of PCOS.

7. The invention of claim 6 wherein the compound of formula (I) is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one.

8. The invention of claim 6 wherein the compound of formula (I) is 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one (1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine).

9. The invention of claim 6 wherein the compound of formula (I) is 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one.

10. A kit comprising a pyrazolopyrimidinone PDE5 inhibitor according to any of the preceding claims in an effective amount and one or more of:
a. means for testing for PCOS.
b. one or more pharmaceutically acceptable carrier, excipient or diluent
c. one or more additional active agents.

11. A compound, composition, method or use substantially as described herein.

12. A phamaceutical composition comprising sildenafil and an additional active agent and optionally a pharmaceutically acceptable carrier.

13. A pharmaceutical composition according to claim 12 wherein the additional agent in metformin or clomid.
